(19) United States
(12) Patent Application Publication
(10) Pub. No.: US 2021/0322509 A1
(43) Pub. Date: Oct. 21, 2021

(54) Methods for Reducing Perinatal Morbidity and / or Mortality

(71) Applicants: David OLSON, Edmonton (CA); CHU SAINTE-JUSTINE, Montreal (CA); UNIVERSITÉ DE MONTRÉAL, Montreal (CA); THE UNIVERSITY OF ADELAIDE, Adelaide (AU)

(72) Inventors: Sylvain CHEMTOB, Côte St-Luc (CA); Christiane QUINIOU, Montreal (CA); Sarah Anne ROBERTSON, College Park (AU); Mathieu NADEAU-VALLÉE, Montreal (CA); William D. LUBELL, Montreal (CA); David OLSON, Edmonton (CA); Peck Yin CHIN, Campbelltown (AU); Sylvie GIRARD, Montréal (CA); Doan-Ngoc DUC, Cambridge, MA (US)

(73) Assignees: CHU SAINTE-JUSTINE, Montreal (CA); UNIVERSITÉ DE MONTRÉAL, Montreal (CA); THE UNIVERSITY OF ADELAIDE, Adelaide (AU); David OLSON, Montreal (CA)

(21) Appl. No.: 17/364,557

(22) Filed: Jun. 30, 2021

Related U.S. Application Data
(63) Continuation of application No. 16/082,922, filed on Sep. 6, 2018.

(51) Int. Cl. A61K 38/08; C07K 7/06; A61P 29/00

(52) U.S. Cl. CPC A61K 38/08; A61K 38/00; A61P 29/00; C07K 7/06

(57) ABSTRACT

A method for preventing or reducing the risk of perinatal or neonatal morbidity and mortality caused by antenatal inflammation in humans is described. This method is based on the administration of a compound of formula I or a pharmaceutically acceptable salt thereof:

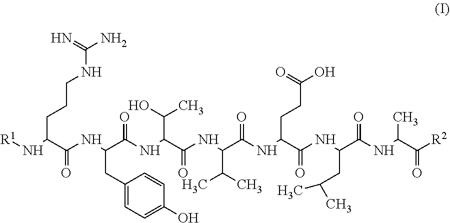

to an expectant human mother suffering from antenatal fetal inflammation. The method prevents or reduces the risk of organ damages, including brain, lung and intestinal damages, and sequelae therefrom, in the neonates, as well as the risk of neonatal death.

### CROSS-REFERENCE TO RELATED APPLICATIONS

The present application is a Continuation Application of U.S. application Ser. No. 16/082,922 filed on Sep. 6, 2018, which is a National Entry Application of PCT application No. PCT/CA2016/050253 filed on Mar. 9, 2016. The content of all of the above applications is incorporated herein by reference in its entirety.

### SEQUENCE LISTING

A sequence listing is submitted herewith as an ASCII compliant text file named 12810_801-Seq listing_ST25.txt, that was created on Jun. 29, 2021 and having a size of ˜ 4 kilobytes. The content of the aforementioned file hereby incorporated by reference in its entirety.

### TECHNICAL FIELD

The present invention generally relates to neonatalogy, and more specifically to the prevention of perinatal/neonatal morbidity and/or mortality associated with maternal inflammation.

### BACKGROUND ART

The greatest risk of childhood death occurs during the neonatal period, which extends from birth through the first month of life. About 60 percent of deaths to children under age 5 and nearly two-thirds of infant deaths (birth to 12 months) occur during the neonatal period (Rutstein, 2000), and about two-thirds of all neonatal deaths occur during the first week of life. Current estimates place the annual neonatal death toll at 4 million (Save the Children, 2001).

Normal fetal development and growth typically occur in a sterile amniotic cavity (or at least an amniotic cavity free of pathogenic microorganisms), and first exposure to microorganisms happens at birth. However, perinatal morbidity and mortality often occur in mothers with microbial invasion of the amniotic cavity and associated inflammation. Microbial attack of the fetus takes place in approximately 10% of pregnancies with intra-amniotic infection. The human fetus is capable of deploying an inflammatory response (cellular and humoral) in the mid-trimester of pregnancy, which leads to secretion of pro-inflammatory cytokines such as interleukin Interleukin-1 beta (IL-1p) and tumor necrosis factor alpha (TNF-alpha). These cytokines are also produced by intrauterine tissues in response to microbial products. Systemic and placental maternal infections (e.g., urinary tract infections, chorioamnionitis) occurring at the end of gestation are recognized as triggers of perinatal inflammation. Such maternal infections are believed to be mainly due to bacterial microorganisms, Escherichia coli being one of the most prevalent, but in most instances the infectious cause is sub-clinical and only manifested by the inflammatory component.

Maternal infection/inflammation is one of the major independent risk factors for perinatal brain lesions, both in premature and term newborns, and also increases the risk of fetal death (Grether, J. K., and K. B. Nelson. 1997. JAMA 278: 207-211; Wu, Y. W., and J. M. Colford, Jr. 2000. JAMA 284: 1417-1424; Shalak, L. F., 2002. Pediatrics 110: 673-680). A fetal inflammatory systemic response occurs in a fraction of fetuses exposed to microorganisms in utero, and is associated with the impending onset of labor as well as multisystem organ involvement. Neonates born with funisitis, one of the histologic marker of such inflammation, are at increased risk for perinatal organ damages, neurologic handicap, cerebral palsy (Nelson, K. B. and Chang, T, Curr. Opin. Neurol. 21: 129-135), respiratory distress, gastro-intestinal dysfunction, visual and hearing handicap, for which few effective preventive or therapeutic interventions are available; moreover, antibiotics have not been shown to be effective in alleviating adverse perinatal outcome (Kenyon et al., 2001. Lancet 357(9261):979-88) other than preventing Group B Streptococcal infections. Antenatal infection/inflammation is also associated with an enhanced susceptibility to diseases and conditions occurring later and is likely to inflict noxious fetal imprints that program the development of some severe neuropsychiatric illnesses, such as schizophrenia and autism, in progeny (Meyer, U et al., J. Neurosci. 26: 4752-4762; Smith, S. E., et al., J. Neurosci. 27: 10695-10702). Improved obstetrical and neonatal care has fallen short of the hope of reducing the incidence of perinatal neurologic handicaps associated with maternal inflammation/infection. Likewise, currently used tocolytics have not been shown to improve neonatal outcomes, such as neonatal mortality.

Thus, there is a need for the development of novel approaches to reduce neonatal mortality and morbidity associated with maternal infection and/or inflammation.

The present description refers to a number of documents, the content of which is herein incorporated by reference in their entirety.

### SUMMARY OF THE INVENTION

The present invention provides the following items 1 to 64:

1. A compound of formula I (SEQ ID NO:1), or a pharmaceutically acceptable salt thereof:

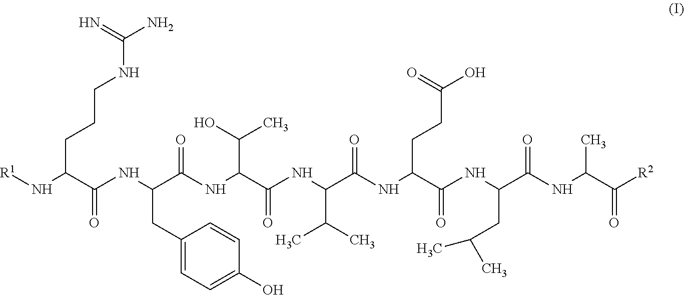

wherein:
R¹ is H or a C₁-C₁₂ alkyl or acyl group;
R² is OH or NR³R⁴, wherein R³ and R⁴ are each independently H or C₁-C₃ alkyl;
for use in the prevention or reduction of the risk of perinatal or neonatal morbidity and mortality caused by antenatal fetal inflammation, wherein said compound or pharmaceutically acceptable salt thereof is for administration to an expectant mother suffering from antenatal fetal inflammation.
2. The compound for use according to item 1, wherein R¹ is H.
3. The compound for use according to item 1 or 2, wherein R² is OH.
4. The compound for use according to item 1 or 2, wherein R² is NH₂.
5. The compound for use according to any one of items 1 to 4, wherein a compound of formula Ia (SEQ ID NO:2), or a pharmaceutically acceptable salt thereof, is used:

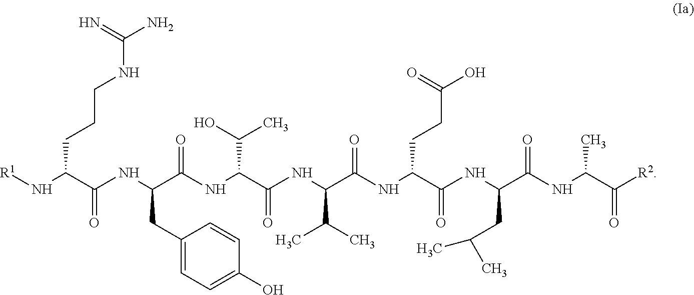

6. The compound for use according to item 5, wherein the following compound, or a pharmaceutically acceptable salt thereof, is used:

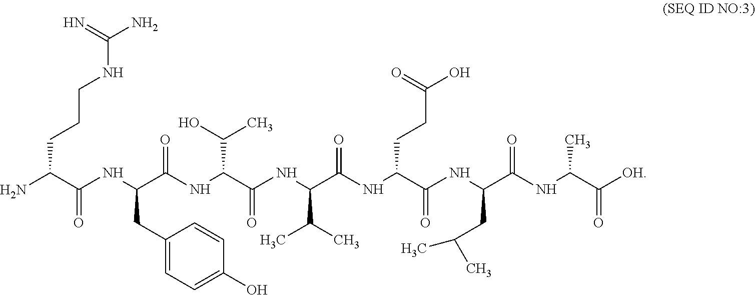 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(=O)O]

7. The compound for use according to item 6, wherein the following compound, or a pharmaceutically acceptable salt thereof, is used:

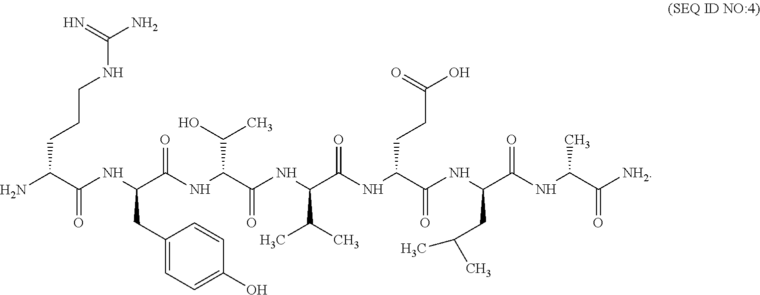 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(N)=O]

8. The compound for use according to any one of items 1 to 7, wherein the antenatal fetal inflammation comprises antenatal intrauterine inflammation.
9. The compound for use according to any one of items 1 to 8, wherein the perinatal or neonatal morbidity comprises organ damage or injury.
10. The compound for use according to item 9, wherein the organ is the lungs, the brain and/or the intestines.
11. The compound for use according to item 10, wherein the organ is the lungs, the brain and the intestines.
12. The compound for use according to any one of items 1 to 11, wherein the perinatal or neonatal morbidity comprises a neurological or neurodevelopmental disorder.
13. The compound for use according to item 12, wherein said neurodevelopmental disorder is cerebral palsy, mental deficiency, or autism.
14. The compound for use according to any one of items 1 to 13, wherein said neonatal death is death within the first week of life.
15. The compound for use according to any one of items 1 to 14, wherein said expectant mother suffers from an infection.
16. The compound for use according to item 15, wherein said infection is uteroplacental infection.
17. The compound for use according to item 15 or 16, wherein said infection is urinary tract infection or intra-amniotic infection.
18. The compound for use according to any one of items 15 to 17, wherein said infection is a bacterial infection.
19. The compound for use according to item 18, wherein said bacterial infection is a gram-negative bacterial infection
20. The compound for use according to item 19, wherein said gram-negative bacterial infection is an Escherichia coli infection.
21. The compound for use according to any one of items 1 to 20, wherein said compound is for injection, oral administration, or fetal administration.
22. A method for preventing or reducing the risk of perinatal or neonatal morbidity and mortality caused by antenatal fetal inflammation, the method comprising administering an effective amount of a compound of formula I (SEQ ID NO:1), or a pharmaceutically acceptable salt thereof, to an expectant human mother afflicted by antenatal fetal inflammation:

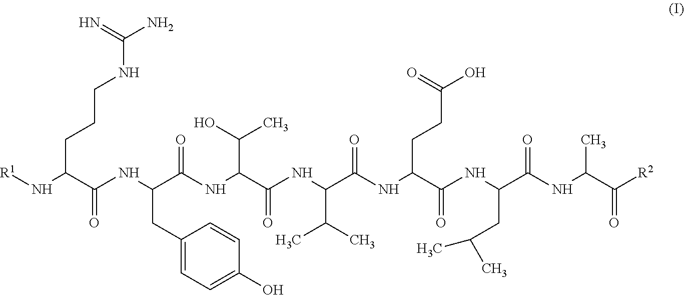

wherein:
R¹ is H, a C₁-C₁₂ alkyl group or a C₁-C₆ acyl group;
R² is OR³ or NR³R⁴, wherein R³ and R⁴ are each independently H or C₁-C₃ alkyl.
23. The method of item 22, wherein R¹ is H.
24. The method of item 22 or 23, wherein R² is OH.
25. The method of item 22 or 23, wherein R² is NH₂.
26. The method of any one of items 22 to 25, wherein said method comprises administering an effective amount of a compound of formula Ia (SEQ ID NO:2), or a pharmaceutically acceptable salt thereof:

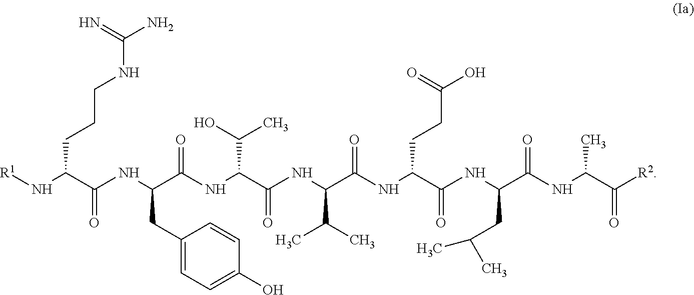

27. The method of item 26, wherein said method comprises administering an effective amount of the following compound, or a pharmaceutically acceptable salt thereof:

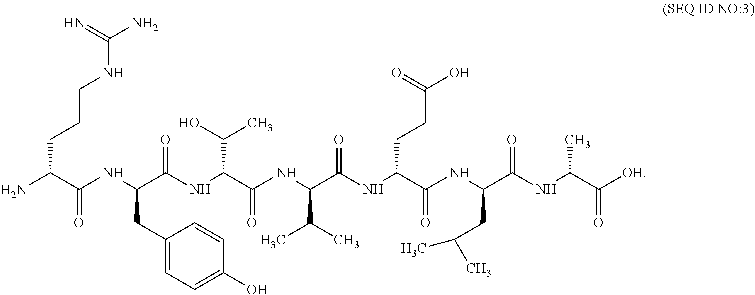 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(=O)O]

28. The method of item 26, wherein said method comprises administering an effective amount of the following compound, or a pharmaceutically acceptable salt thereof:

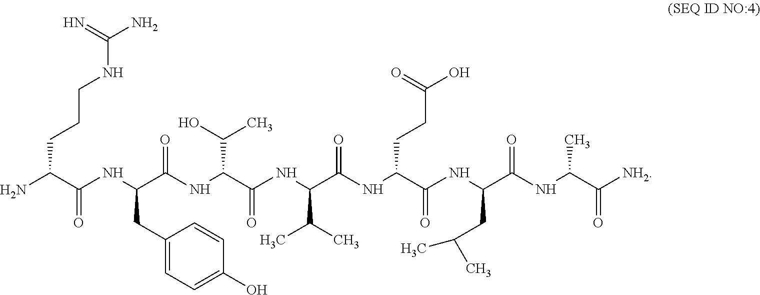 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(N)=O]

29. The method of any one of items 22 to 28, wherein the antenatal fetal inflammation comprises antenatal intrauterine inflammation.
30. The method of any one of items 22 to 29, wherein the perinatal or neonatal morbidity comprises organ damage or injury.
31. The method of item 30, wherein the organ is the lungs, the brain and/or the intestines.
32. The method of item 31, wherein the organ is the lungs, the brain and the intestines.
33. The method of any one of items 22 to 32, wherein the perinatal or neonatal morbidity comprises a neurological or neurodevelopmental disorder.
34. The method of item 33, wherein said neurological or neurodevelopmental disorder is cerebral palsy, mental deficiency, or autism.
35. The method of any one of items 22 to 34, wherein the neonatal mortality is death within the first week of life.
36. The method of any one of items 22 to 35, wherein said expectant mother suffers from an infection.
37. The method of item 36, wherein said infection is uteroplacental infection.
38. The method of item 36 or 37, wherein said infection is urinary tract infection or intra-amniotic infection.
39. The method of any one of items 36 to 38, wherein said infection is a bacterial infection.
40. The method of item 39, wherein said bacterial infection is a gram-negative bacterial infection.
41. The method of item 40, wherein said gram-negative bacterial infection is an Escherichia coli infection.
42. The method of any one of items 22 to 41, wherein said compound is for injection, oral administration, or fetal administration.
43. Use of a compound of formula I (SEQ ID NO:1), or a pharmaceutically acceptable salt thereof:

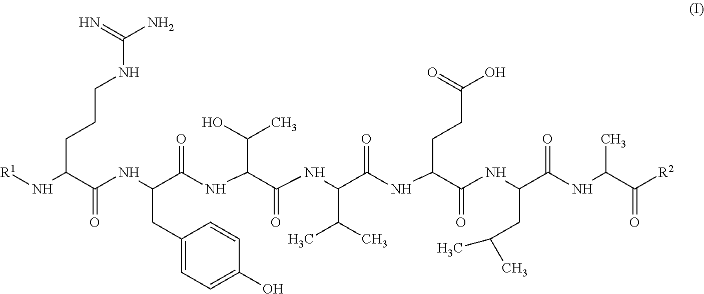

wherein:
R¹ is H or a C₁-C₁₂ alkyl or acyl group;
R² is OH or NR³R⁴, wherein R³ and R⁴ are each independently H or C₁-C₃ alkyl;
for preventing or reducing the risk of perinatal or neonatal morbidity and mortality caused by antenatal fetal inflammation, wherein said compound or pharmaceutically acceptable salt thereof is for administration to an expectant mother suffering from antenatal fetal inflammation.
44. Use of a compound of formula I (SEQ ID NO:1), or a pharmaceutically acceptable salt thereof:

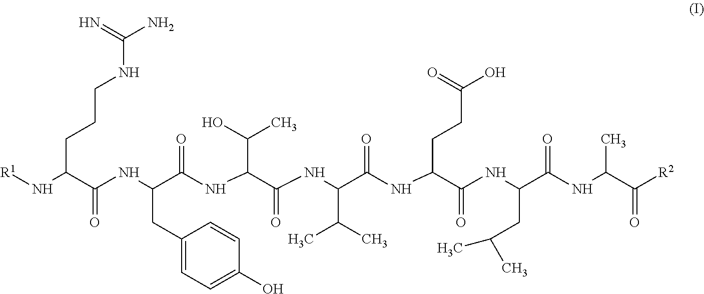

wherein:
R¹ is H or a C₁-C₁₂ alkyl or acyl group;
R² is OR³ or NR³R⁴, wherein R³ and R⁴ are each independently H or C₁-C₃ alkyl;
for preventing or reducing the risk of perinatal or neonatal morbidity and mortality caused by antenatal fetal inflammation, wherein said medicament is for administration to an expectant mother suffering from antenatal fetal inflammation.
45. The use of item 43 or 44, wherein R¹ is H.
46. The use of any one of items 43 to 45, wherein R² is OH.
47. The use of any one of items 43 to 45, wherein R² is NH₂.
48. The use of any one of items 43 to 47, wherein a compound of formula Ia (SEQ ID NO:2), or a pharmaceutically acceptable salt thereof, is used:

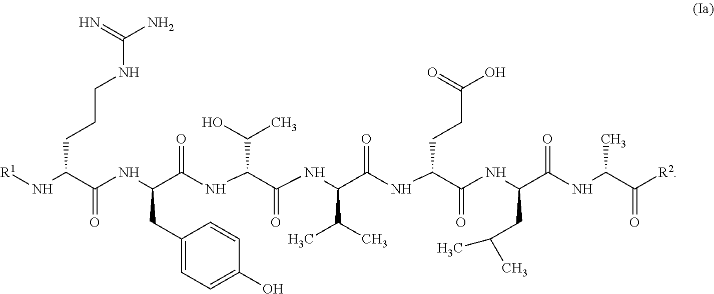

49. The use of item 48, wherein the following compound, or a pharmaceutically acceptable salt thereof, is used:

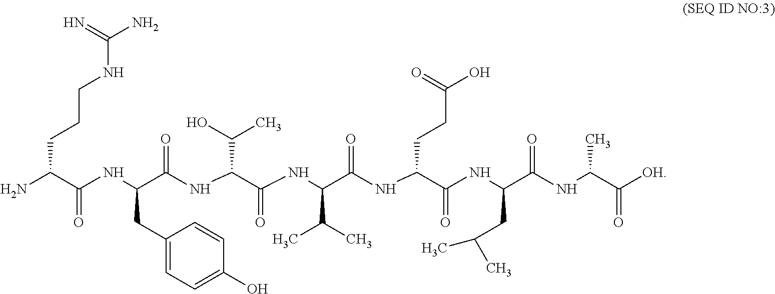 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(=O)O]

50. The use of item 48, wherein the following compound, or a pharmaceutically acceptable salt thereof, is used:

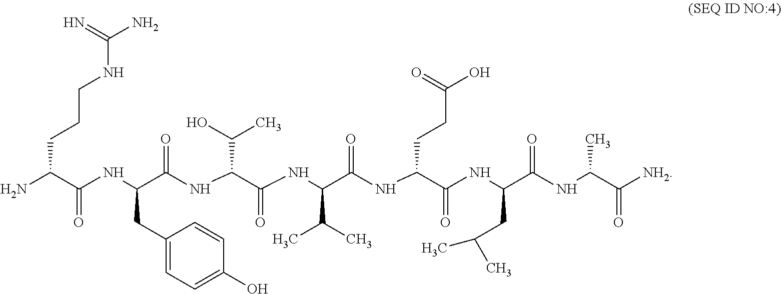 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(N)=O]

51. The use of any one of items 43 to 50, wherein the antenatal fetal inflammation comprises antenatal intrauterine inflammation.
52. The use of any one of items 43 to 51, wherein the perinatal or neonatal morbidity comprises organ damage or injury.
53. The use of item 52, wherein the organ is the lungs, the brain and/or the intestines.
54. The use of item 53, wherein the organ is the lungs, the brain and the intestines.
55. The use of any one of items 43 to 54, wherein the perinatal or neonatal morbidity comprises a neurological or neurodevelopmental disorder.
56. The use of item 55, wherein said neurodevelopmental disorder is cerebral palsy, mental deficiency, or autism.
57. The use of any one of items 43 to 56, wherein said neonatal death is death within the first week of life.
58. The use of any one of items 43 to 57, wherein said expectant mother suffers from an infection.
59. The use of item 58, wherein said infection is uteroplacental infection.
60. The use of item 58 or 59, wherein said infection is urinary tract infection or intra-amniotic infection.
61. The use of any one of items 58 to 60, wherein said infection is a bacterial infection.
62. The use of item 61, wherein said bacterial infection is a gram-negative bacterial infection.
63. The use of item 62, wherein said gram-negative bacterial infection is an Escherichia coli infection.
64. The use according to any one of items 43 to 63, wherein said compound is for injection, oral administration, or fetal administration.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A shows the animal model used in Examples 1 to 5 described herein. Timed-pregnant CD-1 mice were exposed to 1 μg of IL-1β at 16.5 days of gestation (G16.5). Compound 1 (Cmpd 1), Kineret or vehicle was injected subcutaneously in the neck skin 30 minutes before stimulation with IL-1β, and mice delivery was assessed every hour until term (G19-G19.5).

FIG. 1B shows the percentages of neonates' survival at birth in sham and IL-1β-treated administered with vehicle, Compound 1 or Kineret. One-way ANOVA compared to IL-1β+Veh. ***p<0.001. Control (n=9), sham (n=7), IL-1β (n=26), IL-1β+Compound 1 (n=22), IL-1β+Kin (n=11).

FIG. 1C shows caesarean section of dams after a 24 h exposition to intrauterine IL-1β.

FIGS. 2A to 2D show the levels of the pro-inflammatory mediators IL-1β (FIG. 2A), IL-6 (FIG. 2B), IL-8 (FIG. 2C) and PGF2a (FIG. 2D) in the amniotic fluid (AF) collected 24 h after IL-1β injection and treatment with vehicle, Compound 1 or Kineret, as assessed by ELISA. One-way ANOVA compared to IL-1β+Veh. **p<0.01, ***p<0.001. STATS: n=4; 4 gestational sacs from 2 animals in each groups.

FIGS. 3A to 3D show the levels of the pro-inflammatory mediators IL-1p (FIG. 3A), IL-6 (FIG. 3B), IL-8 (FIG. 3C) and PGF2a (FIG. 3D) in the lungs of neonates from sham and IL-1β-treated dams administered with vehicle, Compound 1 or Kineret, as assessed by ELISA. One-way ANOVA compared to IL-1β+Veh. *p<0.05, **p<0.01. n=4 pups in each groups.

FIG. 4A shows the alveola count (per mm²) in pups from sham and IL-1β-treated dams administered with vehicle, Compound 1 or Kineret. One-way ANOVA compared to IL-1β+Veh. *p<0.05, ***p<0.001. Sham (n=6 pups), IL-1β (n=3 pups), IL-1β+Compound 1 (n=6 pups), IL-1β+Kin (n=8 pups).

FIG. 4B shows representative histological analyses of the lungs of a pup from sham and IL-1β-treated dams administered with vehicle, Compound 1 or Kineret.

FIGS. 5A to 5D show the levels of the pro-inflammatory mediators IL-1p (FIG. 5A), IL-6 (FIG. 5B), IL-8 (FIG. 5C) and PGF2a (FIG. 5D) in the intestines of neonates from sham and IL-1β-treated dams administered with vehicle, Compound 1 or Kineret, as assessed by ELISA. One-way ANOVA compared to IL-1β+Veh. *p<0.05. n=4 pups in each groups.

FIG. 6A to 6D show representative histological analyses of the ileum of neonates from sham (FIG. 6A) and IL-1β-treated dams administered with vehicle (FIG. 6B), Compound 1 (FIG. 6C) or Kineret (FIG. 6D). Arrows indicate crypts. Scale, 1000 μM.

FIGS. 7A and 7B show the number and size, respectively, of resident lymph nodes in the colon of neonates from sham and IL-1β-treated dams administered with vehicle, Compound 1 or Kineret. Sham (n=6 pups), IL-1β (n=3 pups), IL-1β+Compound 1 (n=6 pups), IL-1β+Kin (n=8 pups).

FIG. 7C shows representative histological analyses of the colons of pups from sham and IL-1β-treated dams administered with vehicle, Compound 1 or Kineret. Scale, 250 μM.

FIGS. 8A to 8D show the levels of the pro-inflammatory mediators IL-1β (FIG. 8A), IL-6 (FIG. 8B), IL-8 (FIG. 8C) and PGF2a (FIG. 8D) in fetal brain tissue of neonates from sham and IL-1β-treated dams administered with vehicle, Compound 1 or Kineret, as assessed by ELISA. One-way ANOVA compared to IL-1β+Veh. *p<0.05, **p<0.01, ***p<0.001. n=4 pups in each groups.

FIG. 9 shows the results of a behavioral analysis (Open field test) at PT15 in pups from sham and IL-1-treated dams administered with indomethacin (indo), vehicle, Compound 1 or Kineret. One-way ANOVA compared to IL-1β+Veh. *p<0.05. Sham (n=8 pups), IL-1p (n=8 pups), IL-1β+Compound 1 (n=8 pups), IL-1β+Kin (n=5 pups), IL-1β+indo (n=8 pups).

FIGS. 10A to 10D show the induction of pro-inflammatory cytokines in fetal brain after maternal LPS administration is suppressed by compound 2 (cmpd2) in mice. C57Bl/6 mice were mated to males of the same genotype, administered LPS with or without compound 2 and fetal head were recovered. Relative expression of Il1a (FIG. 10A), IL6 (FIG. 10B), TNF (FIG. 10C) and IL1B (FIG. 10D) mRNAs were determined in each tissue by qPCR and normalized to Actb. Data are shown as mean±SEM relative gene expression in tissue pooled from two implantation sites with n=6 dams/group. The effect of LPS and compound 2 was analyzed by Kruskal-Wallis and Mann-Whitney U-test. #* indicates significant differences between groups, p<0.05.

FIGS. 11A to 11D show the effect of exposure to compound 2 in utero on gestation length, perinatal survival and birth weight in pups. Pregnant females were given either LPS or PBS control i.p. on gestional day (gd) 16.5, then compound 2 or PBS vehicle i.p. at 12 h intervals on gd 16.5, 17.0, 17.5 and 18.0. The timing of birth (FIG. 11A); the number of viable pups per litter at birth (FIG. 11B); the proportion of pups surviving to one week (FIG. 11C), and birth weight at 12-24 h (FIG. 11D) were recorded (all mean±SEM). The number of treated dams in each group is shown in parentheses (n=10 per group, with n=48-57 pups weighed per group at birth) and data were analysed by ANOVA and post-hoc Sidak t-test; *P<0.05.

FIGS. 12A and 12B show the effect of exposure to compound 2 in utero on growth trajectory in offspring. Pregnant females were given either LPS or PBS control i.p. on gd 16.5, then compound 2 or PBS vehicle i.p. at 12 h intervals on gd 16.5, 17.0, 17.5 and 18.0. Growth trajectories of male (FIG. 12A) and female (FIG. 12B) progeny of mothers receiving LPS and/or compound 2 are shown as estimated marginal means±SEM, of n=20 male and n=20 female offspring in each group; *P<0.05). Data was analyzed by a Mixed Model Linear Repeated Measures ANOVA and post-hoc Sidak test.

### DISCLOSURE OF INVENTION

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Unless otherwise defined, all technical and scientific terms used herein have the same meaning as commonly understood by one of ordinary skill in the art to which this invention belongs.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

The use of any and all examples, or exemplary language (“such as”, “e.g.”, “for example”) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Herein, the term “alkyl” has its ordinary meaning in the art. It is to be noted that, unless otherwise specified, the hydrocarbon chains of these groups can be linear or branched. The term “acyl” refers to a group of formula RCO—, where R represents an alkyl group that is attached to the CO group with a single bond.

In the studies described herein, the present inventors have shown in mouse models of antenatal fetal inflammation/infection that administration of Interleukin-1 receptor (IL-1R) antagonists of formula I (compound 1 or 2 as described herein) to the expectant mother is associated with an improved perinatal/neonatal outcome, i.e. prevents or reduces perinatal/neonatal morbidity and/or mortality caused by the inflammation. Notably, administration of the IL-1R antagonists during gestation resulted in a reduction of inflammation-mediated damages to certain organs (lungs, brain and intestines) in the neonates, as well as in a reduction of perinatal/neonatal death, relative to control vehicle-treated animals. Also, compound 1 was significantly more effective that recombinant human interleukin-1 receptor antagonist (Anakinra, trade-name KINERET®) at reducing neonatal death, inflammation and inflammation-mediated damages in neonatal organs.

Accordingly, in a first aspect, the present invention provides a method for improving perinatal/neonatal outcome, for example for reducing perinatal/neonatal morbidity and/or mortality (e.g., associated with antenatal fetal inflammation such as intrauterine inflammation or inflammation of the uterus mucosa), the method comprising administering an effective amount of a compound of formula I (SEQ ID NO:1), or a pharmaceutically acceptable salt thereof, to an expectant human mother in need thereof:

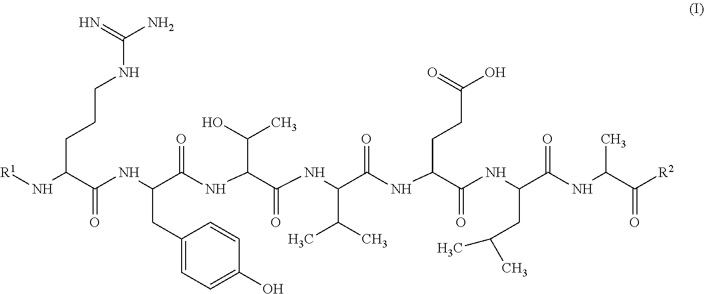

wherein:
R¹ is H or a C₁-C₁₂ alkyl or acyl group, for example a C₁-C₆ alkyl or acyl group, or a C₁-C₃ alkyl or acyl group;
R² is OR³ or NR³R⁴, wherein R³ and R⁴ are each independently H or C₁-C₃ alkyl.

In an embodiment, the alkyl is a linear alkyl.

The term “perinatal/neonatal morbidity” as used herein refers to a disorder or condition in the fetus or neonate, which occurs as a result of adverse influences or treatments acting on the fetus during pregnancy and/or the neonate during the first four weeks of life.

“Antenatal” as used herein refers to the period between conception and birth.

“Perinatal” as used herein refers the period occurring “around the time of birth”, for example, from about 22 completed weeks (154 days) of gestation to about 7 completed days after birth. The postnatal period begins immediately after the birth of a child and then extends for about six weeks. “Neonatal” is defined as a newborn which is an infant who is within seconds, minutes, hours, days, or up to a few weeks from birth. In medical contexts, newborn or neonate refers to an infant in the first month of life (for example about 1, 2, 3 or 4 weeks old). The term “newborn” includes premature infants, postmature infants and full term newborns. In an embodiment, the newborn is a postmature infant or a full term newborn.

In another aspect, the present invention provides a method for improving neonatal outcome, for example for reducing neonatal morbidity and/or mortality (e.g., associated with antenatal fetal inflammation such as intrauterine inflammation or inflammation of the uterus mucosa), the method comprising administering an effective amount of a compound of formula I, or a pharmaceutically acceptable salt thereof, to a newborn in need thereof, e.g., a newborn of a mother who experienced antenatal fetal inflammation.

In an embodiment, perinatal/neonatal morbidity comprises organ damages, including damages to the lungs, the brain and/or the intestines, respiratory problems (asphyxia, bronchopulmonary dysplasia, pneumonia), immune system problems, gastrointestinal problems (e.g., necrotizing enterocolitis), systemic and pulmonary hypertension, early onset neonatal sepsis, septic shock, and/or neurological or developmental problems/handicaps. The neurological and/or developmental problems in the newborns may result in short-, mid- and/or long-term neurological conditions, complications or sequelae, such as cerebral palsy, impaired cognitive skills, behavioral and psychological problems (e.g., mental deficiency and autism). Thus, the term “reducing perinatal/neonatal morbidity” or “reducing the risk of perinatal/neonatal morbidity” encompasses reducing direct damages, injuries and disorders of the fetus or newborn, but also long-term complications/sequelae thereof that may occur later during childhood/adulthood (or reducing the risk of developing such disorders/complications).

In an embodiment, the above-mentioned method is for reducing neonatal morbidity and/or mortality.

In another aspect, the present invention provides a method for preventing or reducing the risk of neonatal organ damages, neonatal neurodevelopmental disorder and/or neonatal death in a human newborn, the method comprising administering an effective amount of a compound of formula I, or a pharmaceutically acceptable salt thereof, to an expectant human mother in need thereof.

In an embodiment, the above-mentioned method is for preventing or reducing neonatal organ damages. In another embodiment, the above-mentioned method is for preventing or reducing neonatal brain damages, as well as reducing the risk of suffering from neurodevelopmental or psychological disorder.

In another aspect, the present invention provides a method for preventing or reducing the risk of neonatal death of a human newborn, the method comprising administering an effective amount of a compound of formula I, or a pharmaceutically acceptable salt thereof, to an expectant human mother in need thereof.

As used herein, the term “preventing” refers to the reduction, in a statistical sample, of the occurrence of the disorder or condition in the treated sample relative to an untreated control sample, the delay of the onset and/or the reduction of the severity of one or more symptoms of the disorder or condition relative to the untreated control sample.

In an embodiment, the expectant human mother in need of treatment exhibits, or is at risk or suspected of exhibiting, antenatal fetal inflammation, e.g., (fetal inflammatory response syndrome (FIRS), urinary tract infection, amniotic-fluid inflammation, intrauterine, and/or uteroplacental perinatal inflammation, i.e. inflammation with pathophysiological IL-1 (IL-1β) synthesis/secretion.

In an embodiment, the expectant human mother is infected with an infectious agent that promotes inflammation and pathophysiological IL-1 (IL-1β) synthesis/secretion, for example bacteria, viruses and other microbes such as yeasts, fungus, as well as parasites such as protozoans and helminth (e.g., candida infection, toxoplasmosis, etc.). Infection as used herein also encompasses microbiome imbalance, e.g., pathophysiological levels of “normal” microbes (or overgrowth of an organism that is present normally at lower levels) that results in inflammation.

In a further embodiment, the expectant human mother exhibits, or is at risk or suspected of exhibiting intraamniotic infection/inflammation (chorioamnionitis) or infection. Chorioamnionitis is often causes by ascending polymicrobial bacterial infection in the setting of membrane rupture, but may also occur with intact membranes in cases of infections with genital mycoplasmas such as Ureaplasma species (e.g., Ureaplasma urealyticum) and Mycoplasma hominis, found in the lower genital tract of a significant proportion of women. Other common isolates in women with intraamniotic infection/inflammation include anaerobes such as Gardnerella vaginalis and bacteroides, as well as aerobes including Group B streptococcus and gram-negative rods including Escherichia coli. In an embodiment, the expectant human mother suffers from a gram-negative bacterial infection.

Clinical signs/symptoms of intraamniotic infection/inflammation include, for example, maternal fever, maternal tachycardia (e.g., >100 BPM) and fetal tachycardia (e.g., >160 BPM), uterine fundal tenderness, vaginal infection and a foul odor to the amniotic fluid (see, e.g., Tita and Andrews, 2010. Clin Perinatol 37(2): 339-354). Laboratory tests on amniotic fluid such as microbial growth, Gram stain, glucose levels, Interleukin-6 levels, presence of matrix metalloproteinase (MMP), white blood cell count and leukocyte esterase, may be useful for the diagnosis of intraamniotic infection/inflammation. Maternal laboratory parameters such as maternal leucocytosis (variously defined as WBC>12,000/mm³ or >15,000/mm³) as well as high levels of C-reactive protein (CRP), lipopolysaccharide binding protein (LBP), soluble intercellular adhesion molecule 1 (sICAM1) and interleukin 6. Placental inflammation may also be detected by magnetic resonance imaging (MRI)-based methods (Girardi G., J Reprod Immunol. 2015 Jul. 2. pii: S0165-0378(15)00094-7).

In an embodiment, the expectant human mother in need of treatment exhibits, or is at risk or suspected of, exhibiting FIRS. FIRS is characterized by systemic inflammation, activation of the fetal immune system, funisitis and increased pro-inflammatory cytokine levels (e.g., IL-6) in umbilical cord blood.

In another embodiment, the expectant human mother has a history of inflammation-related pregnancy complications, or a predisposition to suffering from inflammation-related pregnancy complications.

In an embodiment, the above-mentioned methods further comprises identifying an expectant human mother in need of treatment, i.e. an expectant human mother exhibiting, or at risk or suspected of exhibiting, antenatal fetal inflammation, e.g., intrauterine antenatal inflammation or inflammation of the uterus mucosa.

In an embodiment, the administration of the compound of formula I is initiated preventively, i.e. prior to the development/onset of inflammation (prophylactic treatment). In another embodiment, the administration of the compound of formula I is initiated after the development/onset of inflammation (therapeutic treatment).

The compounds of formula I are antagonists of Interleukin-1 receptor (IL-1R). The synthesis and characterization of these compounds are described in U.S. Pat. No. 8,618,054. These compounds may be readily synthesized by manual and automated solid phase procedures well known in the art. Suitable syntheses can be performed for example by utilizing “t-Boc” or “Fmoc” procedures, segment condensation or other methods known in the art (see, e.g., Behrendt R, J Pept Sci. 2016 January; 22(1):4-27; Hansen and Oddo, Methods Mol Biol. 2015; 1348: 33-50; Amblard et al., Molecular Biotechnology July 2006, Volume 33, Issue 3, pp 239-254; W. D. Lubell, J. W. Blankenship, G. Fridkin, and R. Kaul (2005) “Peptides.” Science of Synthesis 21.11, Chemistry of Amides. Thieme, Stuttgart, 713-809.). These compounds may be synthesized using the methods and conditions disclosed in Example 4 below for compound 2.

Compounds according to formula I, such as compound 1 and 2 described below, or pharmaceutically acceptable salts thereof, may also be purchased from providers of custom chemical synthesis services such as ELIM BIOPHARMACEUTICALS®, GENSCRIPT®, SIGMA-ALDRICH® and the like.

The compounds of formula I have several asymmetric carbon atoms and can therefore exist in the form of optically pure enantiomers, as racemates and as mixture thereof. The synthesis of optically active forms may be carried out by standard techniques of organic chemistry well known in the art, for example by resolution of the racemic form by recrystallisation techniques, by chiral synthesis, by enzymatic resolution, by biotransformation or by chromatographic separation.

In an embodiment, the compound or pharmaceutically acceptable salt thereof is a compound of formula Ia (SEQ ID NO:2), or pharmaceutically acceptable salt thereof:

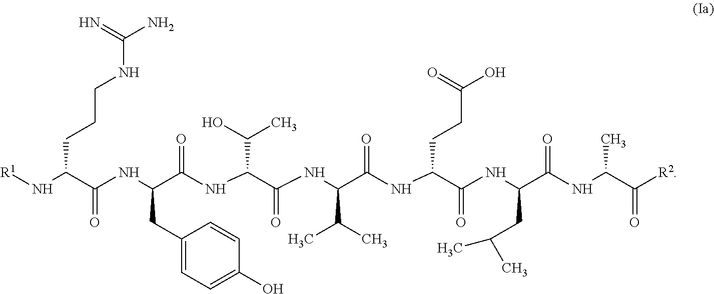

In an embodiment, R¹ is H. In another embodiment, R² is OH or NH₂.

In a further embodiment, the compound or pharmaceutically acceptable salt thereof is compound 1 (cmpd 1) or a pharmaceutically acceptable salt thereof:

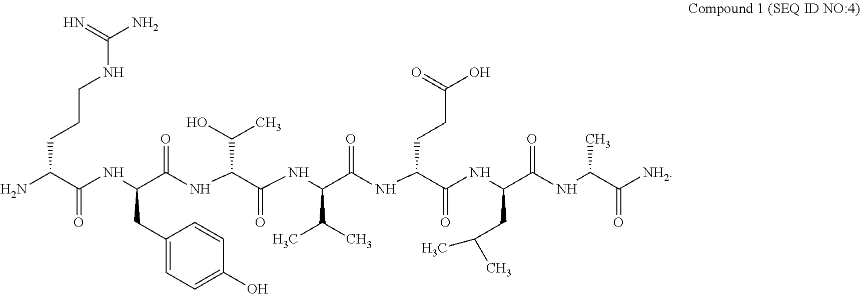 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(N)=O]

In another embodiment, the compound or pharmaceutically acceptable salt thereof is compound 2 (cmpd 2) or a pharmaceutically acceptable salt thereof:

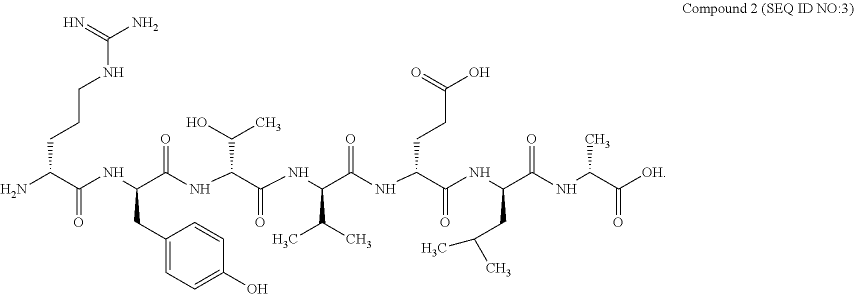 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(=O)O]

Within the present invention, it is to be understood that the compound of formula I may exhibit the phenomenon of tautomerism and that the formulae drawings within this specification can represent only one of the possible tautomeric forms. It is to be understood that the invention encompasses the use of any tautomeric form and is not to be limited merely to any one tautomeric form utilized within the formulae drawings. It is also to be understood that certain compounds may exhibit polymorphism, and that the invention encompasses the use of all such forms.

Also, in embodiments, the above-mentioned compound is in the form of a pharmaceutically acceptable salt. As used herein the term “pharmaceutically acceptable salt” refers to salts of compounds that retain the biological activity of the parent compound, and which are not biologically or otherwise undesirable. Such salts can be prepared in situ during the final isolation and purification of the analog, or may be prepared separately by reacting a free base function with a suitable acid. The above-mentioned compounds are capable of forming acid and/or base salts by virtue of the presence of amino and/or carboxyl groups or groups similar thereto.

Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, decanoate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, octanoate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, formic acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Basic addition salts also can be prepared by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary and tertiary amines. In an embodiment, the the above-mentioned compound is in the form of a formate salt, hydrochloride (HCl) salt, or sodium (Na) salt.

In embodiments, the compounds or pharmaceutically acceptable salts thereof defined herein are comprised in a pharmaceutical composition that also comprises one or more pharmaceutically acceptable carriers and/or excipients. Such compositions may be prepared in a manner well known in the pharmaceutical art. Supplementary active compounds can also be incorporated into the compositions. The carrier/excipient can be suitable, for example, for intravenous, parenteral, subcutaneous, intramuscular, intracranial, intraorbital, ophthalmic, intraventricular, intracapsular, intraspinal, intrathecal, intrauterine, epidural, intracisternal, intraperitoneal, intranasal, rectal, vaginal or pulmonary (e.g., aerosol) administration (see Remington: The Science and Practice of Pharmacy by Alfonso R. Gennaro, 2003, 21^{th} edition, Mack Publishing Company). The compounds or pharmaceutically acceptable salts thereof defined herein are comprised in a pharmaceutical composition formulated in the form of a solution, a tablet, a capsule, a gel/gelatin, a cream, a lotion, a suppository, syrup, an emulsion or a suspension.

In an embodiment, the carrier(s)/excipient(s) is/are suitable for intradermal or subcutaneous administration. In an embodiment, the carrier(s)/excipient(s) is/are suitable for oral administration. In an embodiment, the carrier(s)/excipient(s) is/are suitable for vaginal administration. Therapeutic formulations are prepared using standard methods known in the art by mixing the active ingredient having the desired degree of purity with one or more optional pharmaceutically acceptable carriers, excipients and/or stabilizers.

An “excipient,” as used herein, has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. As those of skill would recognize, a single excipient can fulfill more than two functions at once, e.g., can act as both a binding agent and a thickening agent. As those of skill will also recognize, these terms are not necessarily mutually exclusive.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone (PVP), cellulosic polymers (e.g., hydroxypropyl cellulose (HPC), hydroxypropyl methylcellulose (HPMC), methyl cellulose, hydroxyethyl cellulose, carboxymethylcellulose, colloidal silicon dioxide NF/EP (e.g., Cab-O-Sil™ M5P), Silicified Microcrystalline Cellulose (SMCC), e.g., Silicified microcrystalline cellulose NF/EP (e.g., Prosolv™ SMCC 90), and silicon dioxide, mixtures thereof, and the like), veegum, and combinations thereof.

Useful lubricants include, for example, canola oil, glyceryl palmitostearate, hydrogenated vegetable oil (type 1), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine, calcium stearate, sodium stearyl fumarate, mixtures thereof, and the like.

Bulking agents include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX®).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL®), sodium croscarmelose, sodium starch glycolate (EXPLOTAB®, PRIMO JEL®) crosslinked polyvinylpolypyrrolidone (PLASONE-XL®), sodium chloride, sucrose, lactose and mannitol.

Antiadherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Examples of thickening agent can be for example talc USP/EP, a natural gum, such as guar gum or gum arabic, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; alkyl citrates, used in food packaging, medical products, cosmetics and children toys; triethyl citrate (TEC); acetyl triethyl citrate (ATEC), higher boiling point and lower volatility than TEC; tributyl citrate (TBC); acetyl tributyl citrate (ATBC), compatible with PVC and vinyl chloride copolymers; trioctyl citrate (TOC), also used for gums and controlled release medicines; trihexyl citrate (THC), compatible with PVC, also used for controlled release medicines; acetyl trihexyl citrate (ATHC), compatible with PVC; butyryl trihexyl citrate (BTHC, trihexyl o-butyryl citrate), compatible with PVC; trimethyl citrate (TMC), compatible with PVC; alkyl sulphonic acid phenyl ester, polyethylene glycol (PEG) or any combination thereof.

Examples of permeation enhancers include: sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol and polyethylene glycol), surfactants and terpenes.

Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient (a compound as defined herein) in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for the compounds/compositions described herein include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

For suppositories, the compound (e.g., in powderform) is typically dispersed in a suppository base, such as hard fat. The suppository base can be an oily or fatty base. Conventional suppository bases which may be employed include theobroma oil, hard fats, glycerides of fatty acids, glycerol-gelatin bases, and mixtures thereof. Suitable hard fat bases include, but are no limited to, esterified mixtures of mono-, di- and triglycerides which are obtained by esterification of fatty acids (European Pharmacopoeia, 3^{rd} edition 1997, Deutscher Apotheker Verlag Stuttgart. p. 1022; The United States Pharmacopoeia, USP 23, NF18). Such hard fats are commercially available, for example, under the name Witepsol® (e.g., Witepsol® H12 and H15). Other suitable suppository bases include, but are not limited to, cocoa butter, lauric oil, beef tallow, hard fat, and any combination of any of the foregoing.

Any suitable amount of the compound or pharmaceutical composition may be administered to the expectant mother. The dosages will depend on many factors including the mode of administration. For the prevention, treatment or reduction in the severity of a given disease or condition, the appropriate dosage of the compound/composition will depend on the type of disease or condition to be treated, the severity and course of the disease or condition, whether the compound/composition is administered for preventive or therapeutic purposes, previous therapy, the patient's clinical history and response to the compound/composition, and the discretion of the attending physician. The compound/composition is suitably administered to the patient at one time or over a series of treatments. Preferably, it is desirable to determine the dose-response curve in vitro, and then in useful animal models prior to testing in humans. The present invention provides dosages for the compounds and compositions comprising same. For example, depending on the type and severity of the disease, about 1 μg/kg to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. These are simply guidelines since the actual dose must be carefully selected and titrated by the attending physician based upon clinical factors unique to each patient. The optimal daily dose will be determined by methods known in the art and will be influenced by factors such as the age of the patient and other clinically relevant factors. In addition, patients may be taking medications for other diseases or conditions.

In an embodiment, the compound or composition is administered from week 20 of gestation. In embodiments, the compound or composition is administered from week 20, 21, 22, 23, 24, 25, 26, 27, 28, 29, 30, 31, 32, 33, 34, 35, 36, 37, 38, 39 or 40 of gestation. In an embodiment, the compound or composition is administered before week 37 of gestation. In an embodiment, the compound or composition is administered starting at about week 20-22 of gestation. In an embodiment, the compound or composition is administered starting at about week 23-25 of gestation. In an embodiment, the compound or composition is administered starting at about week 26-28 of gestation.

In an embodiment, the compound or composition is administered starting at about week 29-31 of gestation. In an embodiment, the compound or composition is administered starting at about week 32-34 of gestation. In an embodiment, the compound or composition is administered starting at about week 35-37 of gestation In an embodiment, the above-mentioned treatment comprises the use/administration of more than one (i.e. a combination of) active/therapeutic agent, one of which being the above-mentioned compound of formula I or Ia. The combination of prophylactic/therapeutic agents and/or compositions used in the methods of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) is used/administered in combination with one or more agent(s) currently used to prevent or treat the disorder in question and/or to prevent or treat a related condition. The compounds of formula I or Ia may be co-administered with, for example, tocolytic agents such as β₂-adrenergic receptor agonists or β-mimetics such as Terbutaline (Brethine®, Bricanyl®, Brethaire® or Terbulin®), Ritodrine (Yutopar®), Fenoterol (Berotec N®), Salbutamol/Albuterol (Ventolin®), Ca²⁺ blockers such as Nifedipine (Procardia®, Adalat®), oxytocin receptor antagonists such as Atosiban (Tractocile®, Antocin®, Aatosiban®), Nonsteroidal anti-inflammatory drugs (NSAIDs)/prostaglandin inhibitors such as indomethacin (Indocid®), ketorolac and Sulindac (Clinoril®), Progestin, anti-prostaglandin, nitrates (nitroglycerine) as well as myosin light chain inhibitors such as magnesium sulfate.

The compound(s) may be administered by any routes, for example by intravenous, parenteral, subcutaneous, intramuscular, intracranial, intraorbital, ophthalmic, intraventricular, intracapsular, intraspinal, intrathecal, epidural, intracisternal, intraperitoneal, intrauterine, rectal, vaginal, intranasal or pulmonary (e.g., aerosol) administration (see Remington: The Science and Practice of Pharmacy by Alfonso R. Gennaro, 2003, 21^{th} edition, Mack Publishing Company). Administration to the expectant woman also encompassed delivery of the compound(s) directly to the fetus or gestational tissues in utero. In an embodiment, the compound(s) are administered subcutaneously, i.e. are for subcutaneous administration. In another embodiment, the compound(s) are administered orally, i.e. are for oral administration. In another embodiment, the compound(s) are administered rectally or vaginally, i.e. are for rectal or vaginal administration. In another embodiment, the compound(s) are administered intrauterinally, i.e. are for intrauterine administration. In another embodiment, the compound(s) are administered to the fetus or gestational tissues in utero, i.e. are for fetal administration or administration to gestational tissues.

In another embodiment, the methods and uses defined herein further comprises administration/use of the compound(s) to the neonate, e.g., during the process of birth, immediately after birth, and/or in the postnatal phase. It would be expected that these drugs would have utility after birth, when the consequences of injury before or during birth can become amplified particularly in the event of prematurity and NICU environment.

The compound(s) may be administered at any frequency or according to any dosage regimen, for example once-a-week, twice-a-week, every two days, once-a-day, twice-a-day, etc.

### MODE(S) FOR CARRYING OUT THE INVENTION

The present invention is illustrated in further details by the following non-limiting examples.

### Example 1: Compound 1 Improves Pups Survival in a Perinatal Fetal Inflammation Model

A. Material and Methods

Compounds: Compound 1 was purchased from Elim Biopharmaceuticals (Hayward, Calif.), and Kineret® was purchased from Swedish Orphan Biovitrum AB (Sobi) (Stockholm, Sweden).

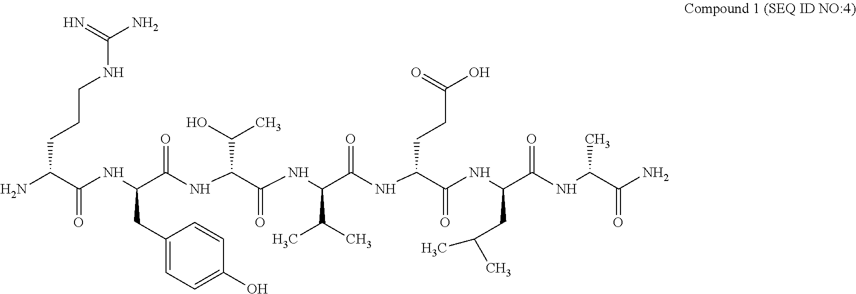 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(N)=O]

Intrauterine IL-1,6-induced perinatal inflammation model. Timed-pregnant CD-1 mice at 16.5 days of gestation were steadily anesthetized with an isoflurane mask. After body hair removal from the peritoneal area, a 1.5 cm-tall median incision was performed with surgical scissors in the lower abdominal wall. The lower segment of the right uterine horn was then exposed and 1 μg of IL-1β was injected between two fetal membranes with care of not entering the amniotic cavity. The abdominal muscle layer was sutured and the skin closed with clips. One hundred μL of Compound 1 (1 mg/Kg/12 h), Kineret® (4 mg/Kg/12 h) or vehicle (sterile water) was injected subcutaneously in the neck 30 minutes before stimulation with IL-1β (to allow distribution of drugs to target tissues). Mice delivery was assessed every hour until term (G19-G19.5). Immediately after delivery, neonatal survival was assessed. Some pregnant mice were sacrificed before birth (24 h after the surgery) to either 1) perform caesarean section and photograph fetuses; 2) collect amniotic fluids; and 3) collect placentas. Only tissues and fluids from gestational sacs closer to the cervical end of the uterus were collected to ensure proximity with the IL-1β injection site. Tissues and fluids were snap-frozen in liquid nitrogen and kept at −80° C. for subsequent RNA purification or protein quantification by ELISA.

B. Results

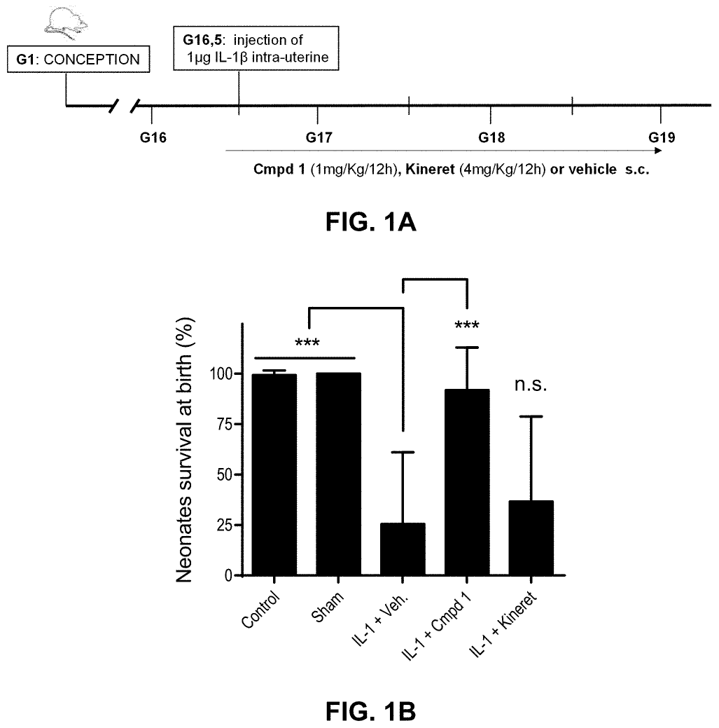

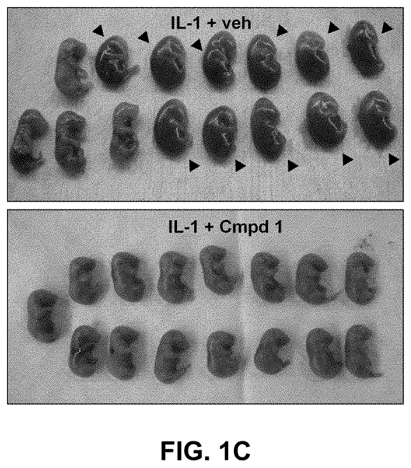

Inflammation during gestation is associated with negative neonatal outcomes. The ability of compound 1 to improve neonatal and developmental outcomes was tested in the setting of a pregnancy threatened by inflammation (induced by administration of 1 μg of IL-1β i.u.), and compared it with Kineret® (anakinra), a recombinant form of the human interleukin-1 receptor antagonist (IL-1Ra) approved by the FDA for the treatment of autoimmune/inflammatory disorders such as rheumatoid arthritis and neonatal-Onset Multisystem Inflammatory Disease (NOMID). As shown in FIG. 1B, compound 1, but not Kineret®, significantly increased neonatal survival in pups from IL-1-treated dams (relative to vehicle). Caesarean section of dams after a 24 h exposition to intrauterine IL-1β shows that 1) treatment with IL-1β induces growth arrest of several fetuses (FIG. 1C, upper panel), and 2) compound 1 prevents this detrimental effect (FIG. 1C, lower panel).

### Example 2: Compound 1 Prevents the Accumulation of IL-1β-Induced Pro-Inflammatory Mediators in the Amniotic Fluid (AF)

A. Material and Methods

Murine ELISA assays. The ELISA assay was performed using mouse Quantikine™ ELISA kits against IL-1β or IL-6 (R&D Systems®; #MLBOOC, M6000B), IL-8 and PGF2α (MyBioSource®; #MBS261967, #MBS264160) according to the manufacturer's instructions. Briefly, 50 μL of either amniotic fluids, recombinant mouse IL-1β, IL-6, IL-8 or PGF2α positive control or decreasing concentrations of a recombinant mouse IL-1β, IL-6, IL-8 or PGF2α standard were loaded into a 96-well plate pre-coated with a monoclonal anti-mouse IL-1β antibody and incubated for 2 hours at ambient temperature. Wells were washed 5 times and incubated with an enzyme-linked mouse polyclonal antibody specific to murine IL-1β for 2 hours. After another washing step, a substrate solution was added. The enzymatic reaction was stopped after 30 minutes and the plate was read at 450 nm, with wavelength correction set to 570 nm.

B. Results

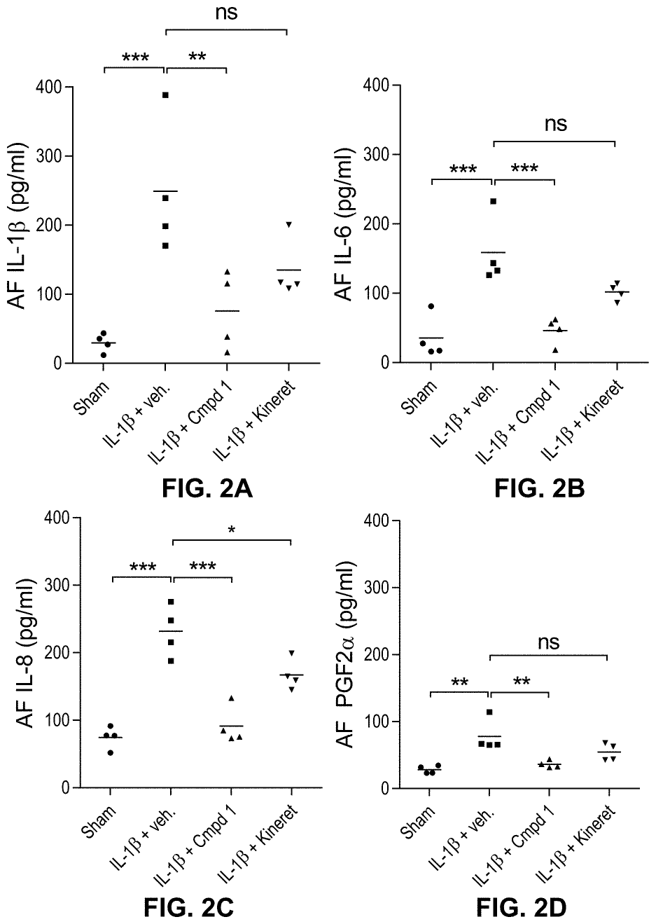

It was assessed whether the inflammation induced in the uterus could affect the fetal environment. An ELISA on amniotic fluids (AF) collected 24 h after the IL-1β injection was performed for each groups. It was found that IL-1β-treated dams had increased levels of pro-inflammatory mediators IL-1β, IL-6, IL-8 and PGF2α in the AF of their concepti (FIGS. 2A-2D), providing evidence that maternal inflammation had driven fetal inflammation. Maternal administration of compound 1 led to a significant reduction of the levels of all pro-inflammatory mediators in the placenta relative to vehicle, whereas Kineret only had a significant effect on IL-8 levels (and at a lower extent relative to compound 1). This significant anti-inflammatory effect of compound 1 on fetal inflammation is consistent with the beneficial neonatal outcomes of this compound shown in FIG. 1B.

### Example 3: Maternal Administration of Compound 1 Decreases Cytokines and Injuries Due to Inflammation in Neonatal Organs

A. Material and Methods

Murine ELISA assays. The ELISA assay was performed using mouse Quantikine™ ELISA kits against IL-1β or IL-6 (R&D Systems®; #MLBOOC, M6000B), IL-8 and PGF2α (MyBioSource®; #MBS261967, #MBS264160) according to the manufacturer's instructions. Briefly, tissues (lungs, intestines and brains) collected at birth were homogenized in RIPA buffer containing proteases and 50 μL of either lung, intestine, or brain samples, recombinant mouse IL-1β, IL-6, IL-8 or PGF2α positive control or decreasing concentrations of a recombinant mouse IL-1β, IL-6, IL-8 or PGF2α standard were loaded into a 96-well plate pre-coated with a monoclonal anti-mouse IL-1β antibody and incubated for 2 hours at ambient temperature. Wells were washed 5 times and incubated with an enzyme-linked mouse polyclonal antibody specific to murine IL-1β for 2 hours. After another washing step, a substrate solution was added. The enzymatic reaction was stopped after 30 minutes and the plate was read at 450 nm, with wavelength correction set to 570 nm.

Histology. Intestines (from ileum to rectum) and lungs of post-term (PT) 15 mice were collected and fixed in 10% formalin during >48 h and were coated in paraffin. Samples were cut with a microtome (thickness=5 μM) and mounted on lamella. Intestines were colored using hematoxylin-phloxine-saffron (HPS), whereas lungs were colored using H&E. Images were taken with a slidescanner (Axioscan®). Quantification of images was performed under blind evaluation of groups using Zen2 software.

Behavioral test (Spontaneous openfield activity). Each animal (PND15 and PND28) was gently placed in the center of the openfield, allowed to freely explore undisturbed for 10 min, after which the animal was removed, the arena cleaned with 70% ethanol and dried prior to testing the next animal. Locomotor activity was indexed as the total distance travelled (cm). The evaluator was blinded to the groups. All movements were monitored and quantified using Smart software.

B. Results

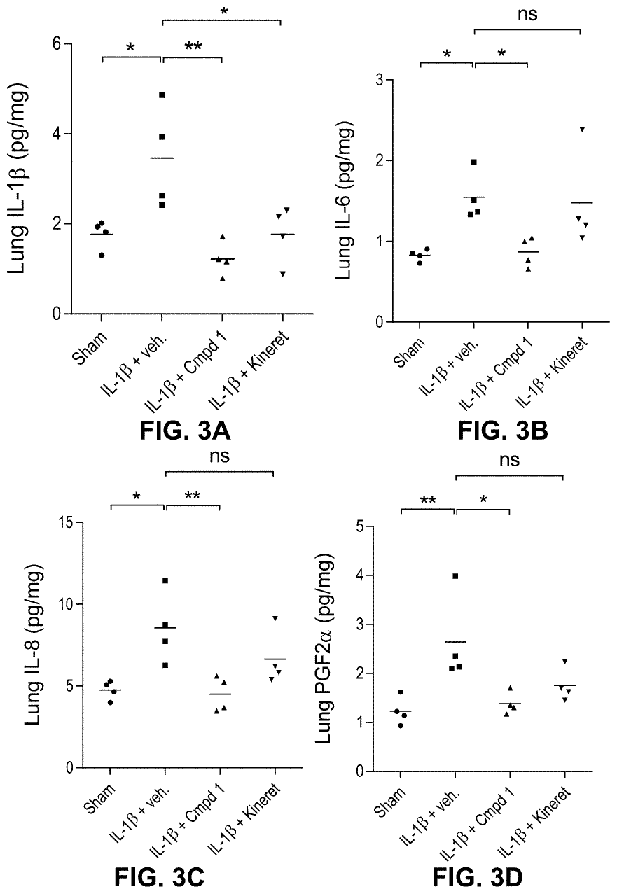

The inflammation in specific tissues known to be afflicted by prematurity, namely the lungs, the intestines and the brain, was assessed. Neonates from IL-1β-treated dams had significantly increased levels of all pro-inflammatory cytokines in their lungs relative to sham, whereas maternal administration of compound 1 resulted in a significant reduction of the levels of all cytokines tested (FIGS. 3A-3D). In contrast, Kineret® was only effective at significantly decreasing lung IL-1β levels (FIG. 3A).

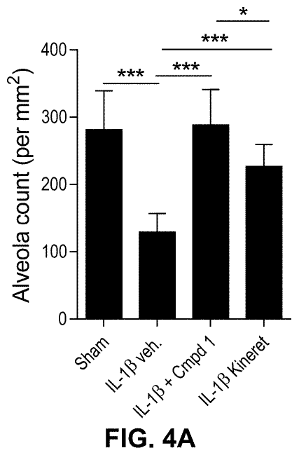

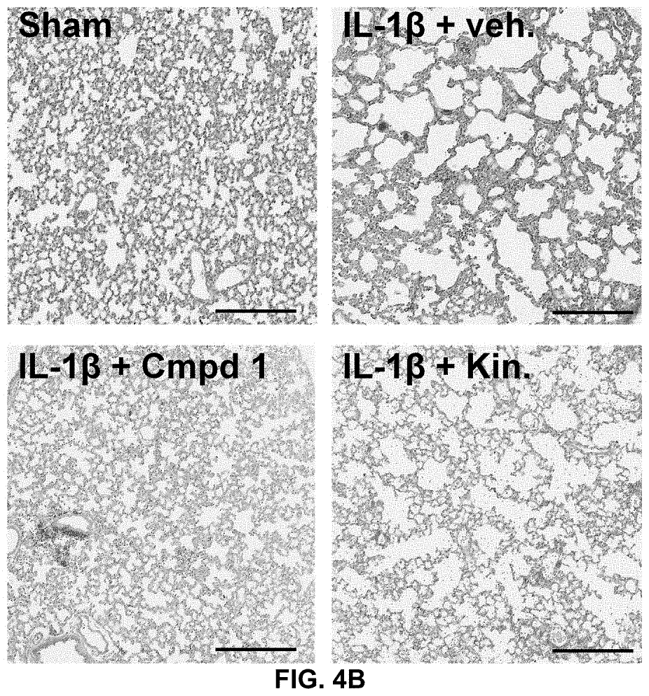

The effect of gestational inflammation on the development of the exposed fetuses was studied. At post-term day (PT) 15, the pups from IL-1β-treated dams were sacrificed, and histological analysis of major organs known to be affected by prematurity/gestational inflammation (the lung, the intestines and the brain) was performed. For the lung, a blind count of alveoli was performed using ImageJ. Pups from IL-1β-treated dams had significantly decreased numbers of alveoli per mm² relative to controls (sham), and treatment with compound 1 completely restored the alveoli count (FIG. 4A), whereas treatment with Kineret® only partially restored the alveoli count. The number of alveoli per mm² in the pups from dams treated with compound 1 was significantly higher than that in pups from dams treated with Kineret®. FIG. 4B shows that treatment with compound 1 prevents the damages to lung architecture caused by IL-1β, and that a more modest effect was observed following treatment with Kineret®.

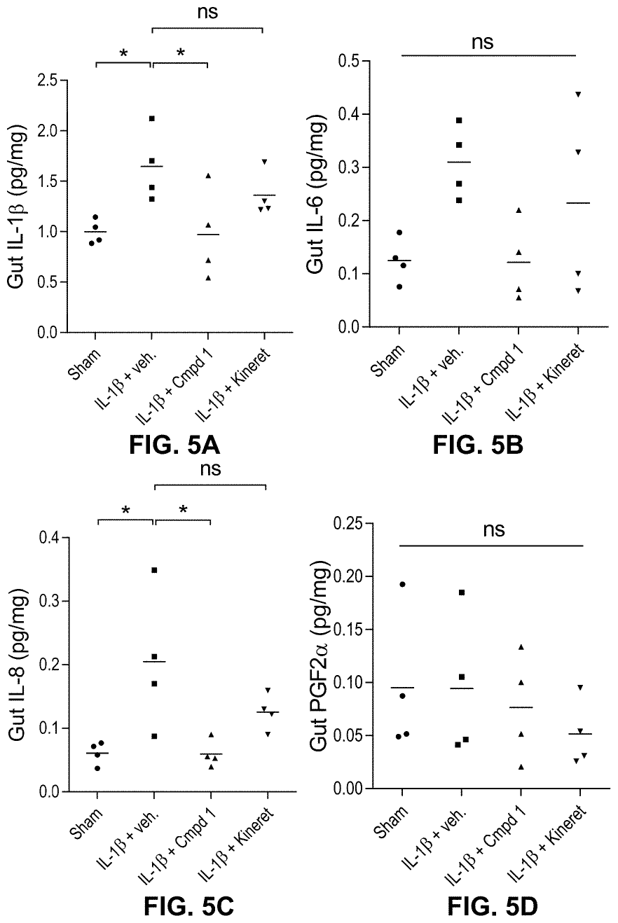

Consistent with the results obtained in the lungs, neonates from IL-1β-treated dams had significantly increased IL-1β and IL-8 in their intestines relative to sham, and maternal administration of compound 1, but not Kineret®, alleviated this effect (FIGS. 5A-5D). There was also a clear trend toward decreased levels of gut IL-6 in the presence of compound 1 relative to control, which was not observed with Kineret®.

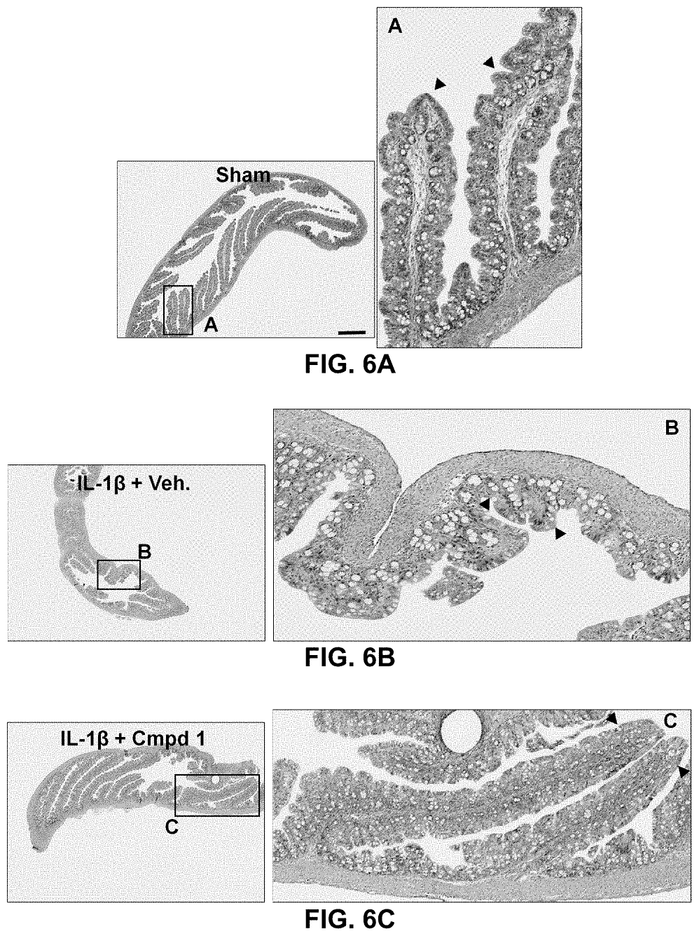

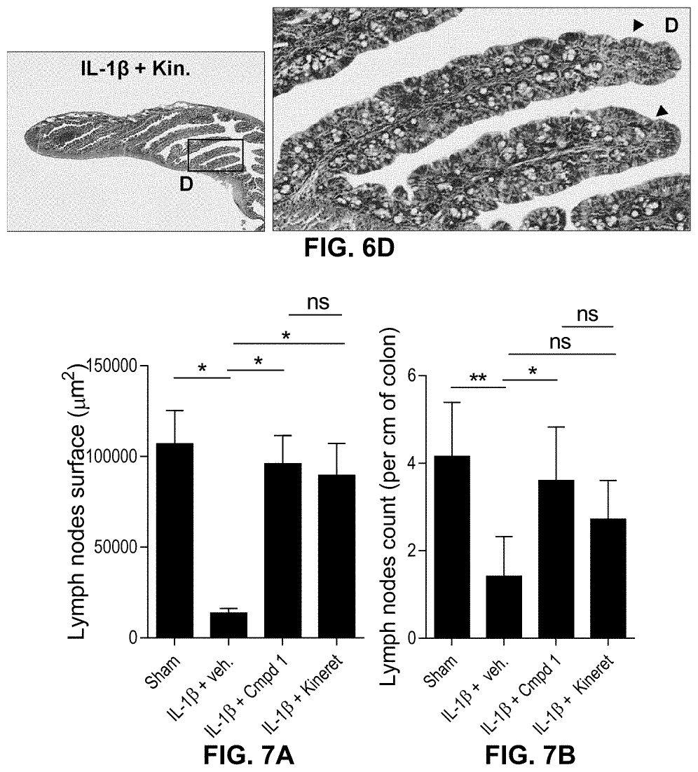

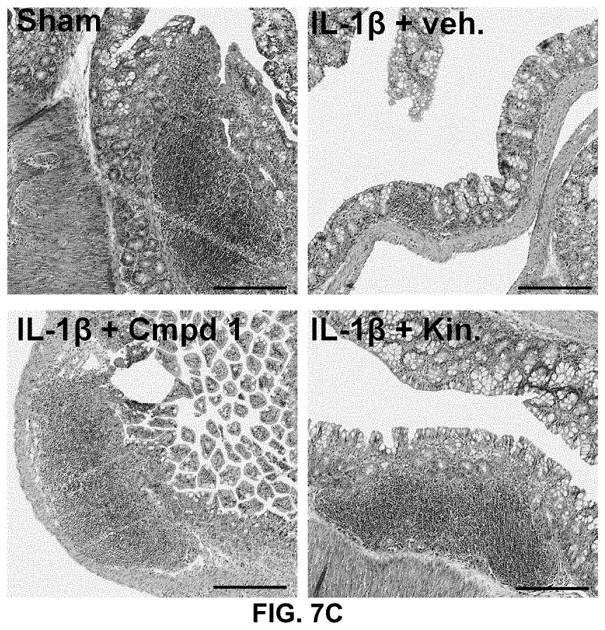

Potential damages to the ileum and in the colon were evaluated by histologic analyses. In the ileum, pups from IL-1β-treated dams administered with vehicle had consistent atrophies in their intestinal crypts (FIG. 6B), and this was not observed following administration of compound 1 (FIG. 6C) or Kineret® (FIG. 6D). In the colon, the number of resident lymph nodes per cm, and their size, were measured. Since lymph nodes are part of the innate immunity and play an important role in the immune surveillance of the colon, the number and size of resident lymph nodes is a marker of the immune integrity/health of the intestine. It was found that pups from IL-1β-treated dams had a significantly reduced number and size of their lymph nodes, which was prevented by administration of compound 1 (FIGS. 7A and 7B). Administration of Kineret® led to a significant increase of the lymph node size relative to vehicle (FIG. 7A), but the lymph node count was not increased at statistically significant levels (FIG. 7B).

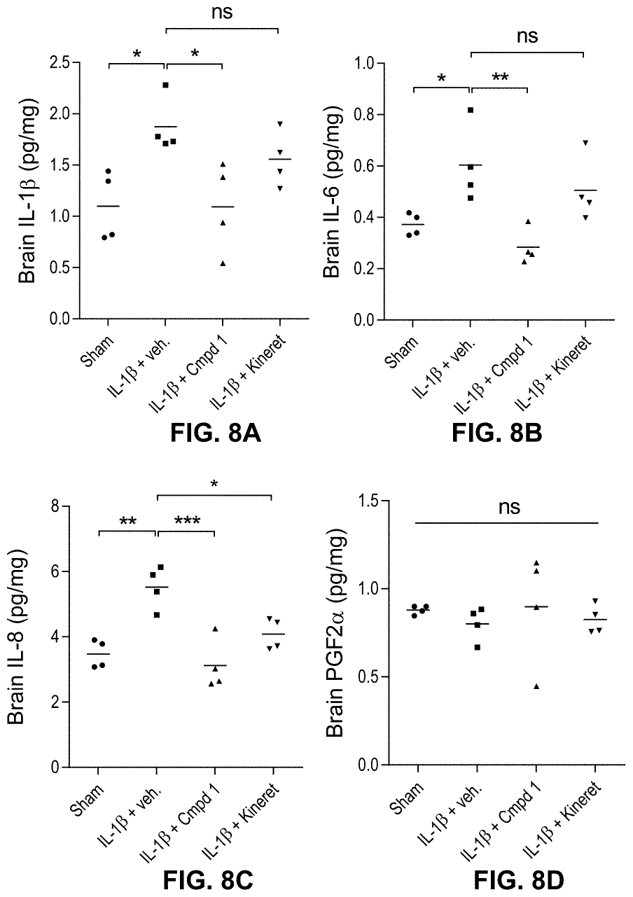

The results depicted in FIGS. 8A-8C, neonates from IL-1β-treated dams had significantly increased IL-1β, IL-6 and IL-8 in their brain relative to sham, and maternal administration of compound 1 alleviated this effect. In contrast, Kineret® was ineffective at significantly reducing the levels of IL-1β and IL-6 in the brain (FIGS. 8A-8B), and the effect on IL-8 levels, although significant, was weaker than that obtained with compound 1 (FIG. 8C).

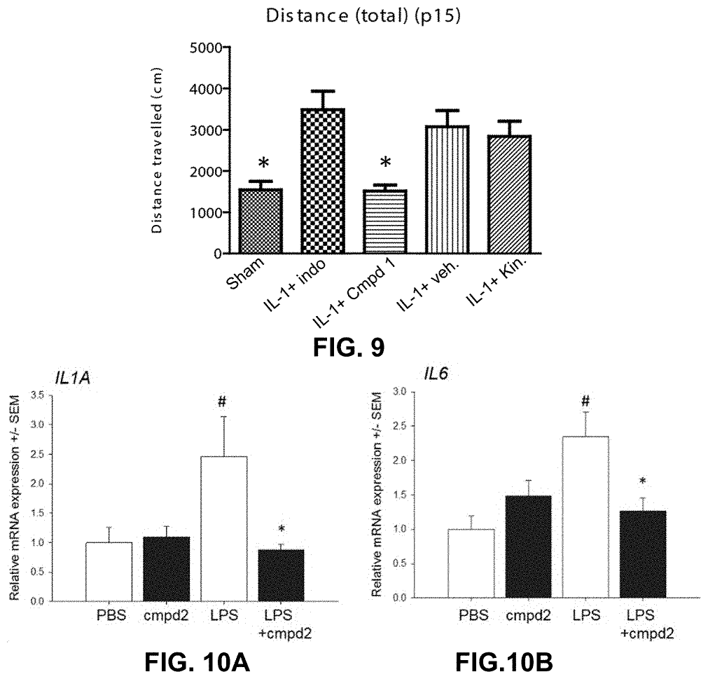

Behavioral tests were performed to assess whether the inflammation in the brain in IL-1β-treated dams led to behavioral/locomotion impairments, and whether such impairments could be reduced or prevented by compound 1. As shown in FIG. 9, pups from IL-1β-treated dams had significant difference in their behavior/locomotion activity relative to sham (i.e., increased distance travelled, which may be an indication of anxiety), and administration of compound 1, but not Kineret® or indomethacin (a commonly used tocolytic agent), led to a normalization of the behavior/locomotion activity (i.e. distance travelled similar to sham).

### Example 4: Maternal Administration of Compound 2 Prevents Inflammation-Induced Cytokine Gene Expression in the Fetal Brain

A. Material and Methods

Compounds: Compound 2 was synthesized and purified as follows.

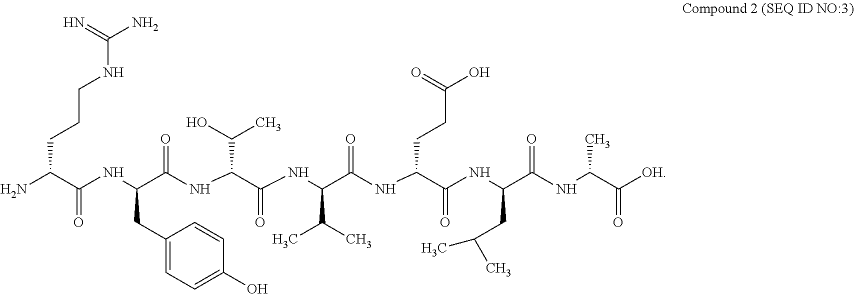 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(=O)O]

In a round bottom flask, 5 g of Wang resin (OH loading: 1.0 mmol/g, 75-100 mesh) was suspended and gently mixed for 30 min in 75 ml of 9:1 v/v dry dichloromethane/dimethylformamide (DCM/DMF). A mixture of 2.34 g of Fmoc-D-Ala-OH (7.5 mmol, 1.5 equiv.) and 1.15 g of Hydroxybenzotriazole (HOBt) (7.5 mmol, 1.5 equiv.), which were dissolved in a minimum amount of dry DMF, was added to the resin. Then, 1.2 mL of Diisopropylcarbodiimide (DIC) (7.5 mmol, 1.5 equiv.), 91 mg of 4-Dimethylaminopyridine (DMAP) (0.75 mmol, 0.15 equiv.), and 100 mL of DCM were subsequently added to the resin mixture. The reaction mixture was mixed overnight on a magnetic stirrer with a metal paper clip as stirrer bar. The resin was filtered and washed 3 times with DMF, MeOH, and then DCM. Unreacted hydroxyl groups on the resin was blocked with 5 mL of acetic anhydride (50 mmol, 10 equiv.) and 8.6 mL of ethyldiisopropylamine (50 mmol, 10 equiv.) for an additional 2 hours at room temperature. The substitution of the resin was spectrophotometrically measured (at 290 nm) to give a final substitution of 0.8 mmol/g.

Syntheses were performed under standard solid phase chemistry conditions (Lubell, W. D. et al.; Science of Synthesis 21.11, Chemistry of Amides., Thieme: Stuttgart, Germany, 2005; pp 713-809) on an automated shaker. Couplings of Fmoc-protected Fmoc-D-Arg(Pbf)-OH, Fmoc-D-Tyr(tBu)-OH, Fmoc-D-Thr(tBu)-OH, Fmoc-D-Val-OH, Fmoc-D-Glu(tBu)-OH, and Fmoc-D-Leu-OH (1.5 equiv) were performed in DMF using (2-(1H-benzotriazol-1-yl)-1,1,3,3-tetramethyluronium hexafluorophosphate) (HBTU) (1.5 equiv) as coupling reagent and N,N-diisopropylethylamine (DIEA) (3 equiv) for 4-8 hours. Fmoc group removal was performed by treating the resin twice with 20% piperidine in DMF for 15 min. The Resin was washed after each coupling and Fmoc-group removal step sequentially with DMF (3×10 mL), MeOH (3×10 mL), THF (3×10 mL), and DCM (3×10 mL). The resin-bound compound 2 was deprotected and cleaved from the support using a freshly made solution of Trifluoroacetic acid/water/Triethylsilane (TFA/H₂O/TES) (95:2.5:2.5, v/v/v, 30 mL/g of peptide resin) at room temperature for 2 h. The resin was filtered and rinsed with TFA. The filtrate and rinses were concentrated until a crude oil persisted, from which a precipitate was obtained by addition of cold ether (10-15 mL). After removing ether, the crude unprotected compound 2 was dried and dissolved in aqueous acetonitrile (10% v/v) and freeze-dried to a white solid, which was analyzed by HPLC to assess purity.

Analyses and characterization of compound 2 were performed on an Agilent™ Technologies 1100 Series LCMS instrument with ESI ion-source, single quadrupole mass detection and positive mode ionization, equipped with a Gilson™ LC 322 pump containing auto-sampler and injector. The LCMS analyses were performed on a Synergi® RP-Polar column (4 μm, 80 Å, 150 mm×4.60 mm I.D.; Phenomenex™, Torrance, USA), using a binary solvent system consisting of 0.1% FA in H₂O, and 0.1% FA in MeOH at a flow rate of 0.5 mL/min and UV detection at 210 nm and 254 nm. Linear gradients of the mobile phase [0-30% methanol (0.1% FA) in water (0.1% FA), over 20 min] were used for analyses of crude compound 2. Preparative RP-HPLC purification of compound 2 samples was conducted using a (4 μm, 80 Å, 150 mm×21.2 mm I.D.; Phenomenex™, Torrance, USA) and an optimized elution gradient (0-5 min. at 0% solvent B, 5-20 min. at 0-20% solvent B, 20-50 min. at 20-25% solvent B, 50-60 min. at 25-60% solvent B, followed by column washing and reconditioning; solvent A: H₂O+0.1% FA; solvent B: MeOH+0.1% FA). Pure fractions were combined and lyophilized to yield a white powder of compound 2.

Animal and treatments. To analyze effects of compound 2 on LPS-induced cytokine expression in the fetal brain after inflammatory stimulus mimicking infection in utero, pregnant female C57Bl/6 female mice (mated with C57Bl/6 males) were injected with 0.5 μg lipopolysaccharide (LPS; Salmonella typhimurium; Sigma-Aldrich®, St. Louis, Mo., USA) in 200 μl PBS i.p., or PBS control, at 1100 h on gd 16.5. Mice were immediately administered compound 2 (1 mg/kg in PBS) or vehicle control (PBS+0.1% BSA) within 5 mins of LPS injection on gd 16.5, then killed 4 h later. Brain tissues were recovered by severing the fetal head from 2 fetuses from each of dams per treatment group.

Expression of inflammatory markers. Messenger RNA (mRNA) was extracted by homogenizing tissues using ceramic beads (Mo Bio) in Trizol® (Ambion® RNA, Carlsbad Calif.). RNA was DNAse-treated using Ambion DNA-Free™ Kit according to the manufacturer's instructions. First strand cDNA was reverse-transcribed from 2 μg extracted RNA with Superscript® III (Invitrogen®, Carlsbad, Calif.) according to the manufacturer's instructions. Primer pairs specific for published cDNA sequences were designed using Primer Express® software (Applied Biosystems®, Foster City, Calif.) to quantify Il1a, Il1b, Il6 and Tnfa mRNA. PCR reactions were performed in a final volume of 20 μL, containing 10 μL of SYBR Green, 7 μL of H₂O, 1 μL each of forward and reverse primers and 1 μL of cDNA template or water (negative/non-template control). PCR conditions were: 10 min at 95° C. followed by 40 cycles of 20 sec at 95° C. and 45 sec at 60° C., using a Rotorgene® 6000 (Corbett Life Sciences®, Sydney, Australia). Data were normalized to β-actin mRNA expression and expressed as ΔΔCT using the formula mRNA level=Log₂−(Ct_{Bacin}−Ct_{target} gene). Statistical analysis was conducted using SPSS for Windows®, version 20.0 software (SPSS Inc, Chicago, Ill.). Data were tested for normality using a Shapiro-Wilk test then analyzed by Kruskal-Wallis and Mann-Whitney U-test since data were not normally distributed. Differences between groups were considered significant when p<0.05.

B. Results

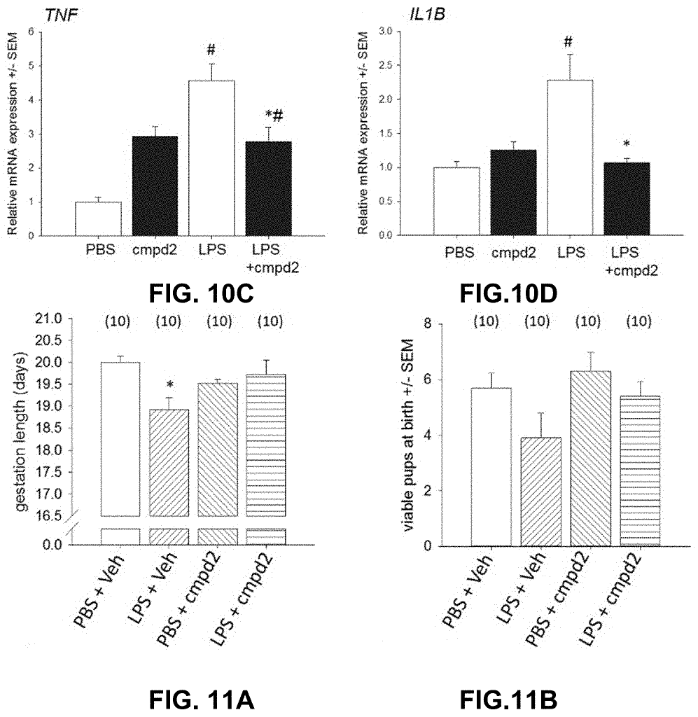

Administration of LPS, which is found in the outer membrane of Gram-negative bacteria and elicits a strong immune response and inflammation in animals (notably through the binding to Toll-Like Receptor 4 (TLR4)), was used to mimic maternal infection-induced inflammation. In fetal head from LPS-treated mice, expression of Il1a (FIG. 10A), IL6 (FIG. 10B), TNF (FIG. 10C) and IL1B (FIG. 10D) was elevated significantly by administration of LPS to dams (p<0.05, FIGS. 9A-D). Induction of all four genes was significantly suppressed when dams were given compound 2 as well as LPS, with expression reduced by 40-60% relative to LPS alone (p<0.05), and no change relative to PBS control for Il1a, Il1b and Il6 (all p<0.05). Compound 2 alone did not significantly alter the expression of Il1a, Il1b and Il6 relative to the PBS control.

### Example 5: Maternal Administration of Compound 2 is Consistent with Normal Postnatal Growth Trajectory and Body Morphometry in Adulthood

A. Material and Methods

Animal and treatments. To analyze effects of compound 2 on perinatal outcomes after inflammatory stimulus mimicking infection in utero, pregnant female C57Bl/6 female mice (mated with C57Bl/6 males) were injected with 0.5 μg lipopolysaccharide (LPS; Salmonella typhimurium; Sigma-Aldrich, St. Louis, Mo., USA) in 200 μl PBS i.p., or PBS control, at 1100 h on gd 16.5. Mice were immediately administered compound 2 (1 mg/kg in PBS) or vehicle control (PBS+0.1% BSA) within 5 mins of LPS injection on gd 16.5, plus a further 3 equivalent doses at 12 h intervals on gd 17.0, 17.5 and 18.0. Mice were monitored via video recording until the time of parturition. Gestation length and perinatal survival (number of live pups at birth and % pups surviving to one week) were recorded. Pups were weighed at 12-24 h after birth.

B. Results

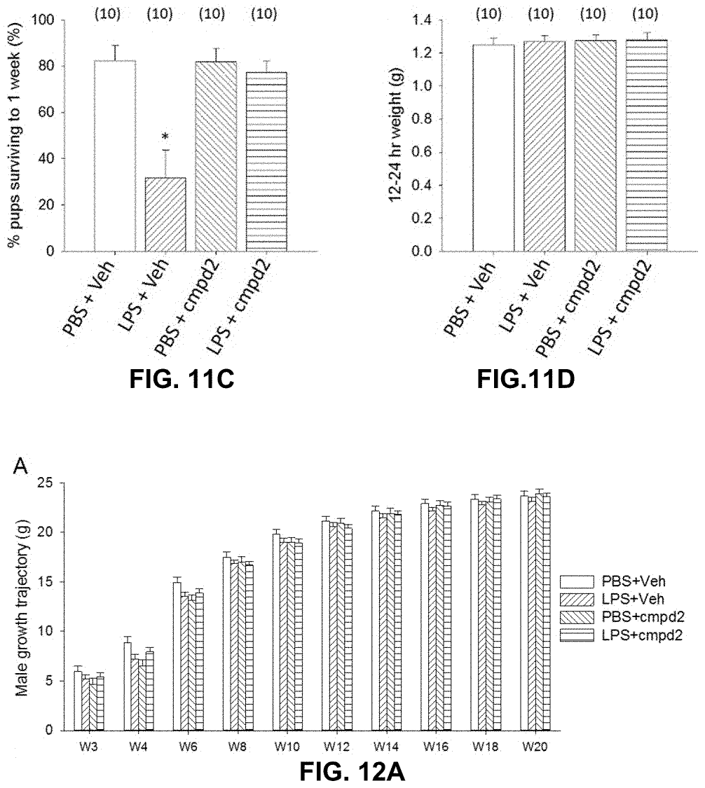

Pregnant mice given LPS without compound 2 exhibited significantly shorter gestation length (FIG. 11A), gave birth to a smaller number of viable pups (FIG. 11B), and a significantly higher rate of pup death in the first week of life was observed (FIG. 11C). Treatment with compound 2 completely reversed the LPS-induced prematurity, perinatal loss, and death in the first week of life (FIGS. 11A-C). Treatment with compound 2 alone, in the absence of LPS, did not alter perinatal outcomes. There was no significant effect of compound 2, with or without LPS treatment, on pup weight at 12-24 h after birth.

### Example 6: Maternal Administration of Compound 2 does not Impede Normal Postnatal Growth Trajectory and Body Morphometry in Adulthood

A. Material and Methods

Animal and treatments. To analyze effects of compound 2 on offspring development into adulthood, pups delivered in the experiment described in Example 5 were weighed at 21 days, when pups were weaned and housed in groups of 1-4 siblings according to sex. All progeny were weighed again at 4 weeks and then every 2 weeks until 20 weeks of age. At 20 weeks, progeny were killed by cervical dislocation, weighed and autopsied for full body composition analysis. The following tissues were excised and weighed individually; brain, heart, lungs (left and right), kidneys (left and right), liver, adrenal glands (left and right), thymus, spleen, testes (males, left and right), seminal vesicle (males), epididymis (males), ovaries (females, left and right), uterus (females), quadriceps (left and right), triceps (left and right), biceps (left and right), gastrocnemius muscle (left and right), retroperitoneal fat, peri-renal fat, epididymal fat (males, left and right) and parametrial fat (females). Weights of bilateral tissues and organs were combined for each mouse. Total muscle weight was calculated by summing the weights of quadriceps, triceps, and biceps and gastrocnemius muscles. Total fat weight was calculated by summing the weights of retroperitoneal fat, peri-renal fat and epididymal fat (for males) or parametrial fat (for females), and the muscle/fat ratio was determined. Total fat weight was subtracted from total body weight to calculate total lean weight.

B. Results

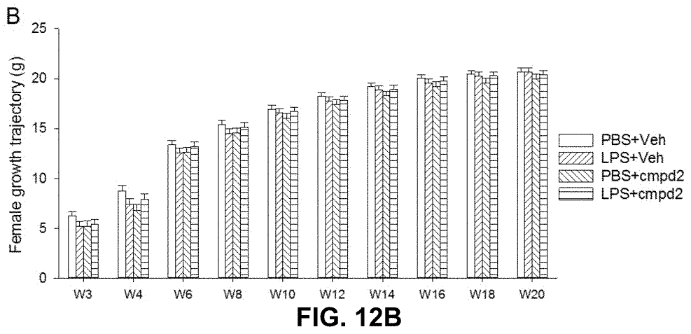

Both male and female offspring of dams exposed to LPS in utero exhibited a growth trajectory indistinguishable from control offspring of dams injected with PBS. Growth trajectory was not affected by administration to dams of compound 2, with or without concurrent LPS exposure (FIGS. 12A and 12B). There was no effect of compound 2 exposure in utero on body composition, other than an increase in spleen weight in adult male offspring seen when compound 2 was given without LPS, but not concurrently with LPS (Tables I and II). A decrease in thymus weight in male offspring of dams exposed to LPS was reversed by concurrent compound 2 administration (Table 1).

TABLE I
Body morphometry in 20 week old adult male progeny after
exposure to LPS and/or compound 2 in utero

| | | | PBS + | LPS + |
|---|---|---|---|---|
| | | | com- | com- |
| Absolute | PBS | LPS | pound 2 | pound 2 |
| weight | N = 0 | N = 10 | N = 16 | N = 22 |
| Lean body | 22.67 ± | 22.73 ± | 23.59 ± | 23.04 ± |
| weight (g) | 0.46 | 0.37 | 0.51 | 0.42 |
| Muscle:fat ratio | 1.30 ± 0.16 | 1.53 ± 0.14 | 1.28 ± | 1.39 ± |
| | | | 0.18 | 0.16 |
| Total Central | 662 ± 42 | 633 ± 33 | 669 ± 46 | 625 ± 37 |
| Fat (mg) | | | | |
| Epididymal Fat | 243 ± 19 | 240 ± 15 | 244 ± 20 | 224 ± 16 |
| (mg) | | | | |
| Retroperitoneal | 347 ± 27 | 318 ± 23 | 353 ± 30 | 334 ± 26 |
| Fat (mg) | | | | |
| Peri-renal Fat | 70 ± 7 | 68 ± 5 | 72 ± 7 | 65 ± 6 |
| (mg) | | | | |
| Combined | 833 ± 21 | 828 ± 17 | 840 ± 23 | 848 ± 18 |
| Muscle (mg) | | | | |
| Gastrocnemius | 249 ± 8 | 249 ± 6 | 241 ± 9 | 260 ± 7 |
| (mg) | | | | |
| Quadriceps | 309 ± 9 | 302 ± 7 | 318 ± 10 | 313 ± 8 |
| (mg) | | | | |
| Biceps (mg) | 58 ± 3 | 63 ± 3 | 67 ± 4 | 58 ± 3 |
| Triceps (mg) | 217 ± 7 | 214 ± 6 | 215 ± 8 | 217 ± 6 |
| Brain (mg) | 414 ± 6 | 409 ± 5 | 404 ± 7 | 413 ± 5 |
| Heart (mg) | 128 ± 3 | 122 ± 3 | 131 ± 4 | 128 ± 3 |
| Lungs (mg) | 170 ± 5 | 167 ± 4 | 181 ± 5 | 173 ± 4 |
| Thymus (mg) | 60 ± 3 | 51 ± 3* | 53 ± 4 | 63 ± 3* |
| Kidneys R | 172 ± 5 | 158 ± 4 | 162 ± 6 | 159 ± 5 |
| (mg) | | | | |
| Kidneys L (mg) | 157 ± 5 | 147 ± 4 | 156 ± 6 | 149 ± 5 |
| Adrenals R | 4 ± 2 | 4 ± 1 | 6 ± 2 | 3 ± 1 |
| (mg) | | | | |
| Adrenals L | 4 ± 0 | 4 ± 0 | 5 ± 0 | 4 ± 0 |
| (mg) | | | | |
| Liver (mg) | 1098 ± 41 | 1051 ± 35 | 1141 ± 47 | 1097 ± 40 |
| Spleen (mg) | 67 ± 5* | 72 ± 4 | 87 ± 5* | 82 ± 4 |
| Seminal | 255 ± 11 | 217 ± 9 | 233 ± 12 | 218 ± 9 |
| Vesicle | | | | |
| (mg) | | | | |
| Testes R (mg) | 87 ± 3 | 85 ± 3 | 87 ± 3 | 85 ± 3 |
| Testes L (mg) | 86 ± 3 | 84 ± 2 | 85 ± 3 | 84 ± 3 |
| Epididymis | 116 ± 3* | 108 ± 2 | 112 ± 4 | 102 ± 3* |
| (L + R) (mg) | | | | |

All data are presented as estimated marginal means ± SEM and analysed as a Mixed Model Linear Repeated Measures ANOVA and post-hoc Sidak test, with litter size as a covariate. *Differences between treatment and control groups were considered significant when of p < 0.05.

TABLE II
Body morphometry in 20 week old adult female progeny after
exposure to LPS and/or compound 2 in utero

| | | | PBS + | LPS + |
|---|---|---|---|---|
| | | | com- | com- |
| | PBS | LPS | pound 2 | pound 2 |
| Absolute weight | N = 22 | N = 11 | N = 14 | N = 11 |
| Lean body | 19.73 ± | 19.91 ± | 19.85 ± | 19.44 ± |
| weight (g) | 0.44 | 0.47 | 0.54 | 0.53 |
| Muscle:fat ratio | 1.19 ± 0.05 | 1.12 ± 0.06 | 1.18 ± 0.06 | 1.23 ± 0.06 |
| Total Central | 623 ± 33 | 629 ± 36 | 589 ± 40 | 562 ± 41 |
| Fat (mg) | | | | |
| Parametrial Fat | 202 ± 16 | 210 ± 18 | 192 ± 20 | 180 ± 20 |
| (mg) | | | | |
| Retroperitoneal | 345 ± 15 | 342 ± 16 | 326 ± 18 | 317 ± 18 |
| Fat (mg) | | | | |
| Peri-renal Fat | 78 ± 6 | 76 ± 7 | 71 ± 7 | 66 ± 7 |
| (mg) | | | | |
| Combined | 731 ± 19 | 692 ± 22 | 690 ± 24 | 673 ± 25 |
| Muscle (mg) | | | | |
| Gastrocnemius | 226 ± 6 | 219 ± 7 | 215 ± 8 | 207 ± 8 |
| (mg) | | | | |
| Quadriceps (mg) | 274 ± 9 | 255 ± 11 | 254 ± 12 | 254 ± 13 |
| Biceps (mg) | 52 ± 3 | 54 ± 3 | 51 ± 3 | 48 ± 3 |
| Triceps (mg) | 180 ± 5 | 164 ± 6 | 170 ± 7 | 165 ± 7 |
| Brain (mg) | 415 ± 7 | 417 ± 8 | 406 ± 9 | 408 ± 9 |
| Heart (mg) | 109 ± 3 | 111 ± 3 | 109 ± 3 | 105 ± 4 |
| Lungs (mg) | 173 ± 9 | 173 ± 9 | 174 ± 11 | 165 ± 10 |
| Thymus (mg) | 64 ± 4 | 60 ± 4 | 64 ± 5 | 70 ± 5 |
| Kidneys R (mg) | 132 ± 5 | 129 ± 5 | 122 ± 6 | 127 ± 6 |
| Kidneys L (mg) | 124 ± 4 | 119 ± 5 | 112 ± 5 | 120 ± 5 |
| Adrenals R (mg) | 4 ± 0 | 4 ± 0 | 4 ± 0 | 4 ± 0 |
| Adrenals L (mg) | 5 ± 0 | 4 ± 0 | 4 ± 0 | 4 ± 0 |
| Liver (mg) | 975 ± 30 | 975 ± 33 | 945 ± 37 | 955 ± 38 |
| Spleen (mg) | 90 ± 6 | 92 ± 6 | 93 ± 7 | 86 ± 7 |
| Uterus (mg) | 64 ± 6 | 72 ± 7 | 63 ± 7 | 66 ± 8 |
| Ovary R (mg) | 16 ± 1 | 14 ± 1 | 16 ± 1 | 13 ± 1 |
| Ovary L (mg) | 17 ± 2 | 16 ± 2 | 16 ± 2 | 13 ± 2 |

All data are presented as estimated marginal means ± SEM and analysed as a Mixed Model Linear Repeated Measures ANOVA and post-hoc Sidak test, with litter size as a covariate. Differences between treatment and control groups were considered significant when of p < 0.05.

The scope of the claims should not be limited by the preferred embodiments set forth in the examples, but should be given the broadest interpretation consistent with the description as a whole.

1-64. (canceled)

65. A method for increasing gestation time in an expectant human mother afflicted by antenatal fetal inflammation, the method comprising administering an effective amount of a compound of formula I (SEQ ID NO:1), or a pharmaceutically acceptable salt thereof, to said expectant human mother: 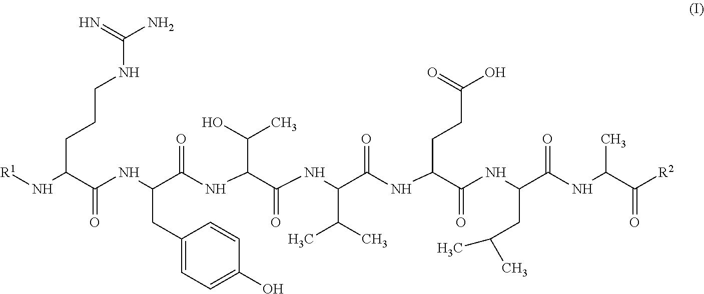
wherein:
R¹ is H, a C₁-C₁₂ alkyl group or a C₁-C₆ acyl group; and
R² is OR³ or NR³R⁴, wherein R³ and R⁴ are each independently H or C₁-C₃ alkyl.

66. The method of claim 65, wherein R¹ is H.

67. The method of claim 65, wherein R² is OH.

68. The method of claim 65, wherein R² is NH₂.

69. The method of claim 65, wherein said method comprises administering an effective amount of a compound of formula Ia (SEQ ID NO:2), or a pharmaceutically acceptable salt thereof:

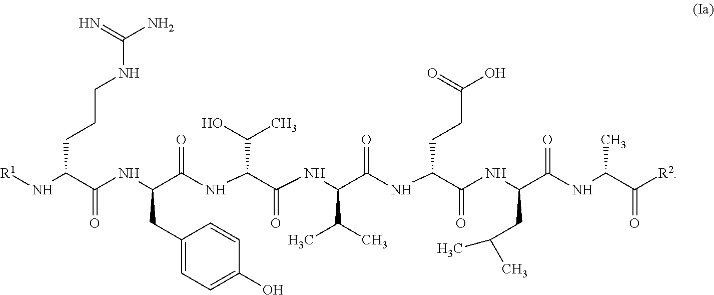

70. The method of claim 65, wherein said method comprises administering an effective amount of the following compound, or a pharmaceutically acceptable salt thereof:

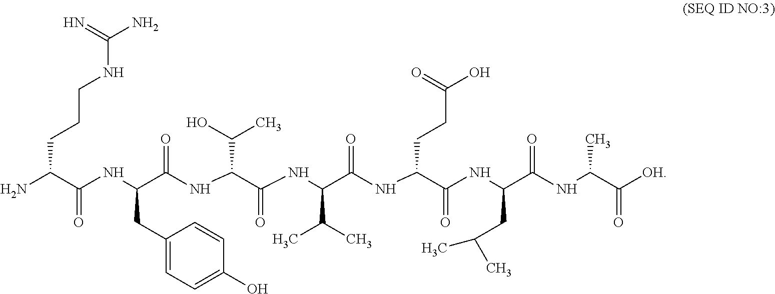 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(=O)O]

71. The method of claim 65, wherein said method comprises administering an effective amount of the following compound, or a pharmaceutically acceptable salt thereof:

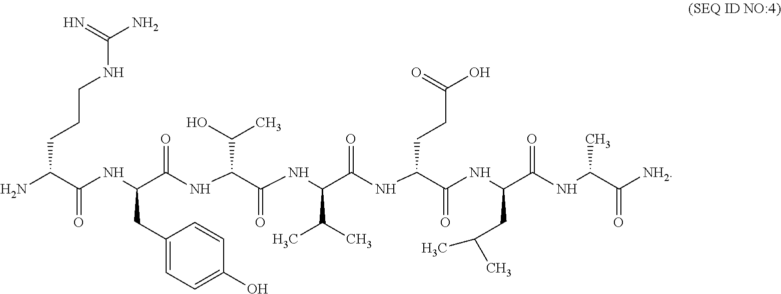 [SMILES: CC(C)C[C@@H](NC(=O)[C@@H](CCC(=O)O)NC(=O)[C@H](NC(=O)[C@H](NC(=O)[C@@H](Cc1ccc(O)cc1)NC(=O)[C@H](N)CCCNC(=N)N)C(C)O)C(C)C)C(=O)N[C@H](C)C(N)=O]

72. The method of claim 65, wherein the method further reduces the risk of neonatal mortality within the first week of life.

73. The method of claim 65, wherein the method further reduces the risk of organ damage or injury in the neonate.

74. The method of claim 73, wherein the organ is brain, lungs and intestines.

75. The method of claim 65, wherein the method prevents premature birth.

76. The method of claim 65, wherein the compound or pharmaceutically acceptable salt thereof is initiated after the development of the antenatal fetal inflammation.

77. The method of claim 65, wherein the expectant mother suffers from an infection.

78. The method of claim 77, wherein the infection is uteroplacental infection.

79. The method of claim 77, wherein the infection is a bacterial infection.

80. The method of claim 79, wherein the bacterial infection is a gram-negative bacterial infection.

81. The method of claim 80, wherein the gram-negative bacterial infection is an Escherichia coli infection.

82. The method of claim 65, wherein the administration is by injection.

83. The method of claim 65, wherein the compound or pharmaceutically acceptable salt thereof is administered starting at about week 20 to 22 of gestation.

84. The method of claim 65, wherein the expectant human mother suffers from fetal inflammatory response syndrome (FIRS).


SEQUENCE LISTING (4 sequences)

SEQ ID NO: 1 - protein, 7 aa, Artificial Sequence
  Synthetic peptide
  MISC_FEATURE (1)..(1): Xaa is D-arg or L-arg
  MISC_FEATURE (1)..(1): The amino-terminal end of the peptide comprises a NHR1 group, wherein R1 is H, a C1-C12 alkyl group or a C1-C12 acyl group;
  MISC_FEATURE (2)..(2): Xaa is D-tyr or L-tyr
  MISC_FEATURE (3)..(3): Xaa is D-thr or L-thr
  MISC_FEATURE (4)..(4): Xaa is D-val or L-val
  MISC_FEATURE (5)..(5): Xaa is D-glu or L-glu
  MISC_FEATURE (6)..(6): Xaa is D-leu or L-leu
  MISC_FEATURE (7)..(7): Xaa is D-ala or L-ala
  MISC_FEATURE (7)..(7): The carboxy-terminal end of the peptide comprises an OH group or a NR3R4 group, wherein R3 and R4 are each independently H or C1-C3 alkyl
XXXXXXX

SEQ ID NO: 2 - protein, 7 aa, Artificial Sequence
  Synthetic peptide
  MISC_FEATURE (1)..(1): Xaa is D-arg
  MISC_FEATURE (1)..(1): The amino-terminal end of the peptide comprises a NHR1 group, wherein R1 is H, a C1-C12 alkyl group or a C1-C12 acyl group;
  MISC_FEATURE (2)..(2): Xaa is D-tyr
  MISC_FEATURE (3)..(3): Xaa is D-thr
  MISC_FEATURE (4)..(4): Xaa is D-val
  MISC_FEATURE (5)..(5): Xaa is D-glu
  MISC_FEATURE (6)..(6): Xaa is D-leu
  MISC_FEATURE (7)..(7): Xaa is D-ala
  MISC_FEATURE (7)..(7): The carboxy-terminal end of the peptide comprises an OH group or a NR3R4 group, wherein R3 and R4 are each independently H or C1-C3 alkyl
XXXXXXX

SEQ ID NO: 3 - protein, 7 aa, Artificial Sequence
  Synthetic peptide
  MISC_FEATURE (1)..(1): Xaa is D-arg
  MISC_FEATURE (2)..(2): Xaa is D-tyr
  MISC_FEATURE (3)..(3): Xaa is D-thr
  MISC_FEATURE (4)..(4): Xaa is D-val
  MISC_FEATURE (5)..(5): Xaa is D-glu
  MISC_FEATURE (6)..(6): Xaa is D-leu
  MISC_FEATURE (7)..(7): Xaa is D-ala
XXXXXXX

SEQ ID NO: 4 - protein, 7 aa, Artificial Sequence
  Synthetic peptide
  MISC_FEATURE (1)..(1): Xaa is D-arg
  MISC_FEATURE (2)..(2): Xaa is D-tyr
  MISC_FEATURE (3)..(3): Xaa is D-thr
  MISC_FEATURE (4)..(4): Xaa is D-val
  MISC_FEATURE (5)..(5): Xaa is D-glu
  MISC_FEATURE (6)..(6): Xaa is D-leu
  MISC_FEATURE (7)..(7): Xaa is D-ala
  MISC_FEATURE (7)..(7): The OH group at the carboxy terminal end is replaced by an NH2 group.
XXXXXXX